(19) United States
(12) Patent Application Publication
(10) Pub. No.: US 2025/0248976 A1
(43) Pub. Date: Aug. 7, 2025

(54) PALATABLE VETERINARY COMPOSITIONS

(71) Applicant: Intervet Inc., Rahway, NJ (US)

(72) Inventors: Sharon Cruz Martin, East Brunswick, NJ (US); Izabela Ewa Galeska, Newtown, PA (US)

(73) Assignee: Intervet Inc., Rahway, NJ (US)

(21) Appl. No.: 18/570,326

(22) PCT Filed: Jun. 24, 2022
(86) PCT No.: PCT/EP2022/067362

§ 371 Date: Dec. 14, 2023

(30) Foreign Application Priority Data
Jun. 25, 2021 (EP) 21181879.4

(51) Int. Cl. A61K 31/42; A61K 9/00; A61K 9/20; A61K 31/365; A61K 31/506

(52) U.S. Cl. CPC A61K 31/42; A61K 9/0056; A61K 9/2009; A61K 9/2013; A61K 9/2031; A61K 9/2095; A61K 31/365; A61K 31/506

(57) ABSTRACT

The present invention is directed to a stable, efficacious and palatable chewable veterinary composition comprising as active ingredient an isoxazoline compound of Formula (I),

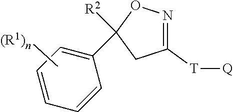

a stabilized macrocyclic lactone, pyrantel and excipients.

### FIELD OF INVENTION

The present application relates to palatable chewable veterinary dosage forms comprising an isoxazoline parasiticidal compound, a physiologically active macrocyclic lactone compound and pyrantel pamoate, to a method for preparing said veterinary dosage form and uses thereof.

### BACKGROUND OF INVENTION

A number of parasites can infest domestic animals, especially companion animals such as cats and dogs. These pests and parasites are of great nuisance to both the animals and their owners.

Isoxazoline compounds are known in the art and these compounds and their use as parasiticides are described, for example, in US patent application US 2007/0066617, and international patent applications WO 2005/085216, WO 2007/079162, WO 2009/002809, WO 2009/024541, WO 2009/003075, WO 2009/080250, WO 2010/070068 and WO 2010/079077.

This class of compounds is known to possess excellent activity against ectoparasites, i.e. parasites that have their permanent or temporary habitat on the external surface of animals, such as parasitic insects and acarids such as ticks and fleas as well as against endoparasites, that parasitize inside an animal's body, e.g. anthelmintics with activity against parasitic nematodes.

One known and convenient way of administering an isoxazoline compound to an animal is oral administration, by way of solid oral dosage forms.

Further, macrocyclic lactones are known to act as very potent parasiticides especially as acaricides, anthelmintic agents and/or insecticides. Thus, they are useful for treating ectoparasites as well as endoparasites of animals.

In view of the above and in order to enhance/improve the therapeutic effect of the isoxazoline compounds, it would be desirable to have a solid oral dosage form further comprising one or more active agents from a different class such as the macrocyclic lactone(s) and pyrantel pamoate in order to broaden the spectrum of parasites controlled by the same dosage form.

However, said active macrocyclic lactones are observed to form a significant amount of degradation product(s). In other words, when added to a common palatable chewable veterinary formulation, macrocyclic lactone(s) are often not stable enough in the resulting dosage form to reliably provide an effective amount of such macrocyclic lactone compound.

EP329460 discloses that the stability of said compounds may be enhanced when they are in the presence of certain antioxidants. In particular, EP329460 discloses the stabilization of moxidectin. The antioxidants described in EP329460 as useful for stabilization are C1. 2 alkyl gallate; C1.6 alkyl hydroxybenzoate or a salt thereof; benzyl hydroxybenzoate or a salt thereof; butylated hydroxyanisole (BHA); butylated hydroxytoluene (BHT); a quinone or a salt thereof; nordihydroguaiaretic acid; and a tocopherol such as a-tocopherol.

AU2006/203347 discloses composition including moxidectin and a stabilizing agent selected from the group consisting of: dilauryl thiodipropionate; monothioglycerol; potassium metabisulfite; sodium formaldehyde sulfoxylate; sodium thiosulfate; thioglycolic acid; thiourea; ascorbyl palmitate; cysteine or a salt thereof; ethoxyquin; isoascorbic acid; ethylene diamine tetra-acetic acid or a salt thereof, potassium bisulfate; sodium metabisulfite; sodium bisulfite; thiosorbitol; fumaric acid; malic acid; and mixtures thereof.

Thus, there is still a need for chewable veterinary dosage forms comprising a combination of an agent from the group of isoxazoline compounds, pyrantel pamoate and a physiologically active macrocyclic lactone compound in which the stability of the physiologically active agents is ensured, and the formation of degradation products of the physiologically active agents is advantageously reduced.

This will advantageously allow a longer shelf life of the resulting product and allows storage of the product under more harsh conditions. Further, the stability of the physiologically active macrocyclic lactone should be improved without having a negative influence on the bioavailability of the physiologically active macrocyclic lactone or the isoxazoline or pyrantel pamoate.

Another important consideration is that the macrocyclic lactone compound is generally present in the palatable chewable veterinary dosage form in a very small amount. This creates a problem with content uniformity, i.e. to make sure that the macrocyclic lactone is uniformly distributed in the tablet.

Hence, it is an object of the present invention to overcome one or more of the drawbacks of the above-mentioned dosage forms.

In particular, it is an object of the present invention to provide a chewable veterinary dosage form inter alia containing a uniform content of physiologically active macrocyclic lactone in a stabilized form such that its degradation is advantageously reduced or preferably even prevented.

Another object is to provide chewable veterinary dosage forms comprising a combination of these three active agents that are effective for the treatment of endo—and ectoparasites in non-human animals and, in particular, of fleas and ticks in small mammals such as dogs and cats that is very palatable to companion animals, especially dogs.

### SUMMARY OF THE INVENTION

The present invention has unexpectedly solved at least one of the above objectives by the provision of a new palatable chewable veterinary dosage form.

Hence, in one aspect the subject of the present invention is directed to a palatable chewable veterinary dosage form in the form of a compressed tablet comprising a) an isoxazoline compound of Formula (I)

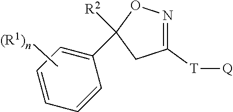

wherein
R¹ is halogen, CF₃, OCF₃, CN,
n is an integer from 0 up to and including 3, preferably 1, 2 or 3,
R² is C₁-C₃-haloalkyl, preferably CF₃ or CF₂Cl,
T is a 5 to 12 membered mono or bicyclic ring system, which is optionally substituted by one or more radicals Y,
Y is methyl, halomethyl, halogen, CN, NO₂, NH₂—C═S, or two adjacent radicals Y form together a chain, especially a three or four-membered chain;
Q is X—NR³R⁴, NR⁵—NR⁶—X—R³, X—R³ or a 5-membered N-heteroaryl ring, which is optionally substituted by one or more radicals;
X is CH₂, CH(CH₃), CH(CN), CO, CS,
R³ is hydrogen, methyl, haloethyl, halopropyl, halobutyl, methoxymethyl, methoxyethyl, halomethoxymethyl, ethoxymethyl, haloethoxymethyl, propoxymethyl, ethylaminocarbonylmethyl, ethylaminocarbonylethyl, dimethoxyethyl, propynylaminocarbonyl-methyl, N-phenyl-N-methyl-amino, haloethylaminocarbonylmethyl, haloethylaminocarbonylethyl, tetrahydrofuryl, methylaminocarbonylmethyl, (N,N-dimethylamino)-carbonylmethyl, propylaminocarbonylmethyl, cyclopropylaminocarbonylmethyl, propenylaminocarbonylmethyl, halo-ethylaminocarbonylcyclopropyl, alkylsulfanyl, alkylsufinalkyl, alkylsulfonalkyl, cycloalkyl

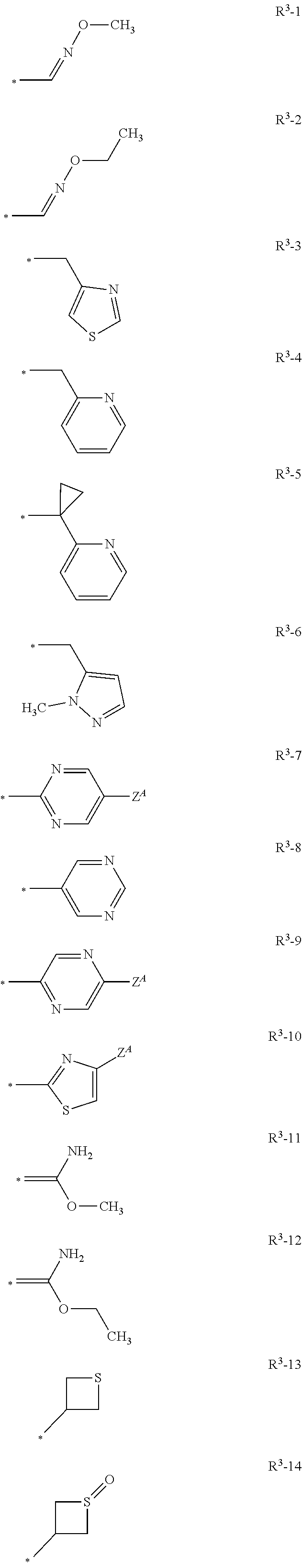

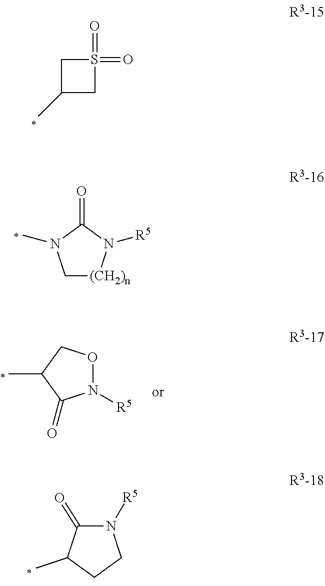

wherein Z^{A} is hydrogen, halogen, cyano, halomethyl, preferably CF₃;
R⁴ is hydrogen, ethyl, methoxymethyl, halomethoxymethyl, ethoxymethyl, haloethoxy-methyl, propoxymethyl, methylcarbonyl, ethylcarbonyl, propylcarbonyl, cyclopropylcarbonyl, methoxycarbonyl, methoxymethylcarbonyl, aminocarbonyl, ethylaminocarbonylmethyl, ethylaminocarbonylethyl, dimethoxyethyl, propynylaminocarbonylmethyl, haloethylaminocarbonylmethyl, cyanomethylaminocarbonylmethyl, or haloethyl-aminocarbonylethyl;
R⁵ is hydrogen, alkyl or haloalkyl;
R⁶ is hydrogen, alkyl or haloalkyl;
or R³ and R⁴ together form a substituent selected from the group consisting of:

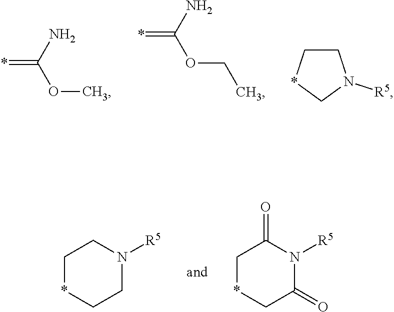

or a salt or solvate thereof, pyrantel pamoate and a macrocyclic lactone compound and a carrier comprising at least one flavor and a stabilizing component that comprises an inorganic alkalizer, porous silica or mixtures thereof.

Also, in a further aspect the invention relates to a process for preparing the palatable veterinary dosage form characterized in that:
a. An isoxazoline and pyrantel pamoate granulation is prepared by: 1) dry blending the isoxazoline compound and pyrantel pamoate with a disintegrant, filler, colorant and surfactant; 2) preparing a solution of a solvent and a cellulosic polymer; 3) blending the isoxazoline and pyrantel pamoate, dry blend with the solution to prepare an isoxazoline-pyrantel granulate in a high-shear mixer granulator; 4) dry and mill the isoxazoline-pyrantel granulate.
b. a moxidectin granulation is prepared by: 1) dry blending a porous silica, a filler and an alkalizer; 2) dissolving moxidectin in a solvent with a non-cellulosic polymer and antioxidant; 4) blending the dry blend with the moxidectin solution to prepare a moxidectin granulate in a high-shear mixer granulator; 4) dry and mill the moxidectin granulate.
c. Preparing a final dry blend by: 1) blending at least one flavor with a filler, a disintegrant, a colorant, a glidant to prepare a mixture; 2) blending the isoxazoline-pyrantel pamoate granulation, and moxidectin granulation with the mixture; 3) blending the mixture with a lubricant and compressing the blend into finished palatable chewable tablets.

Also, in a further aspect the invention relates to the use of the palatable, chewable dosage form according to the invention, or of a palatable, chewable dosage form as obtainable by the method for the preparation of the pharmaceutical composition according to the invention, for the manufacture of a medicament for the treatment or prevention of parasite infestation of animals.

An alternative embodiment is directed to a palatable chewable veterinary dosage form in the form of a compressed tablet comprising an isoxazoline compound of Formula (I)

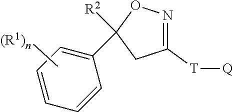

wherein
R¹ is halogen, CF₃, OCF₃, CN,
n is an integer from 0 up to and including 3, preferably 1, 2 or 3,
R² is C₁-C₃-haloalkyl, preferably CF₃ or CF₂Cl,
T is a 5 to 12 membered mono or bicyclic ring system, which is optionally substituted by one or more radicals Y,
Y is methyl, halomethyl, halogen, CN, NO₂, NH₂—C═S, or two adjacent radicals Y form together a chain, especially a three or four-membered chain;
Q is X—NR³R⁴, NR⁵—NR⁶—X—R³, X—R³ or a 5-membered N-heteroaryl ring, which is optionally substituted by one or more radicals;
X is CH₂, CH(CH₃), CH(CN), CO, CS,
R³ is hydrogen, methyl, haloethyl, halopropyl, halobutyl, methoxymethyl, methoxyethyl, halomethoxymethyl, ethoxymethyl, haloethoxymethyl, propoxymethyl, ethylaminocarbonylmethyl, ethylaminocarbonylethyl, dimethoxyethyl, propynylaminocarbonyl-methyl, N-phenyl-N-methyl-amino, haloethylaminocarbonylmethyl, haloethylaminocarbonylethyl, tetrahydrofuryl, methylaminocarbonylmethyl, (N,N-dimethylamino)-carbonylmethyl, propylaminocarbonylmethyl, cyclopropylaminocarbonylmethyl, propenylaminocarbonylmethyl, halo-ethylaminocarbonylcyclopropyl, alkylsulfanyl, alkylsufinalkyl, alkylsulfonalkyl, cycloalkyl

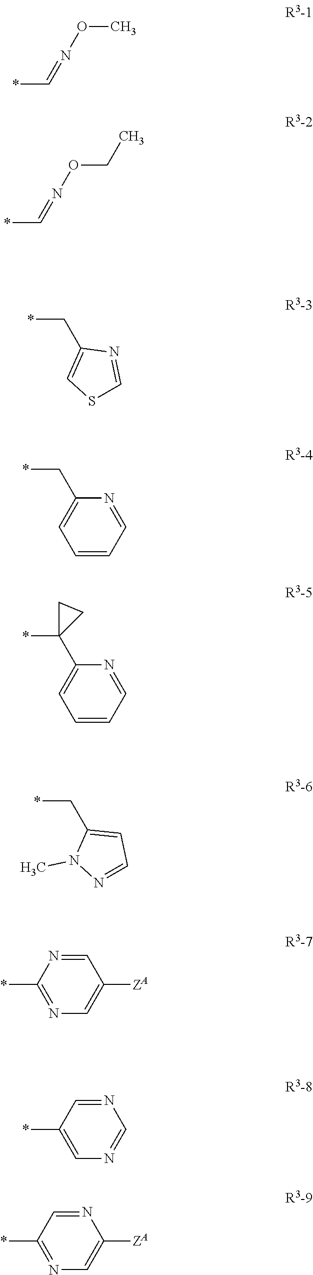

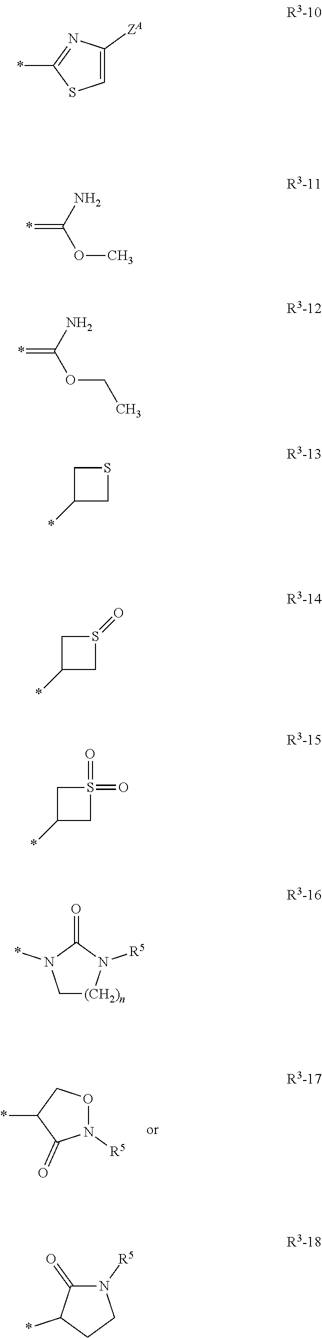

wherein Z^{A} is hydrogen, halogen, cyano, halomethyl, preferably CF₃;
R⁴ is hydrogen, ethyl, methoxymethyl, halomethoxymethyl, ethoxymethyl, haloethoxy-methyl, propoxymethyl, methylcarbonyl, ethylcarbonyl, propylcarbonyl, cyclopropylcarbonyl, methoxycarbonyl, methoxymethylcarbonyl, aminocarbonyl, ethylaminocarbonylmethyl, ethylaminocarbonylethyl, dimethoxyethyl, propynylaminocarbonylmethyl, haloethylaminocarbonylmethyl, cyanomethylaminocarbonylmethyl, or haloethyl-aminocarbonylethyl;
R⁵ is hydrogen, alkyl or haloalkyl;
R⁶ is hydrogen, alkyl or haloalkyl;
or R³ and R⁴ together form a substituent selected from the group consisting of:

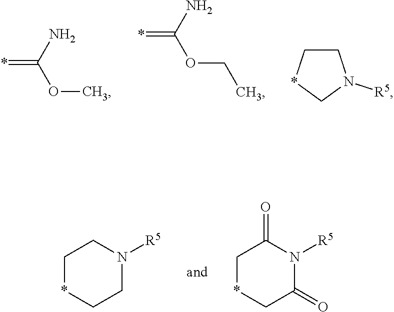

or a salt or solvate thereof, pyrantel pamoate and moxidectin and a carrier comprising at least one flavor and a stabilizing component that comprises a poloxamer more preferably Poloxamer P 188, preferably about 2 to about 15% w/w of the poloxamer.

### DETAILED DESCRIPTION

### Definitions

For purposes of the present invention, as described and claimed herein, the following terms and phrases are defined as follows:

Within the context of invention “veterinary dosage form” refers to a composition containing drugs used to treat and/or diagnose and/or cure and/or prevent diseases of non-human animals. In this application the “veterinary dosage form” is sometimes called “palatable, chewable veterinary dosage form” or “compressed tablet” or “tablet” or “chewable tablet” “finished palatable chewable tablets” or “tablet composition” or alternatively as “composition” or “formulation” or “pharmaceutical composition”. Such composition includes in addition to the stabilising component additionally veterinary acceptable excipients, i.e. an excipient or a combination of excipients (non-active ingredients).

Furthermore, a drug is any substance or combination of substances (composition) presented as having properties for treating or preventing disease in animals; or any substance or combination of substances which may be used in, or administered to animals either with a view to restoring, correcting or modifying physiological functions by exerting a pharmacological, immunological or metabolic action, or to making a medical diagnosis.

According to the FDA glossary, within the context of invention “pharmaceutical composition” or “veterinary dosage form” also refers to a “drug product” which is the finished dosage form that contains a drug substance, generally, but not necessarily in association with other active or inactive ingredients.

According to the present invention the term “veterinary” has the same definition as “pharmaceutical” but adapted to animals (meaning non-human beings).

More precisely, a “veterinary drug” (or medicine or composition) means any substance or mixture of substances which is used, or is manufactured, sold or represented as suitable for use, in the diagnosis, treatment, control, eradication, mitigation or prevention of disease or abnormal physical or mental state or the symptoms thereof in an animal; or restoring, correcting, controlling, or modifying any physical, mental or organic function in an animal.

For use in the invention, an approximate numerical value described as ‘about x’, refers to a value of x with a margin that is ±10% around that value. Alternatively, ±9, 8, 7, 6, 5, 4, 3, 2, or 1% around that value.

In line with the present application the terms “weight %” and “(w/w) %” can be used synonymously and designates weight/weight. As used herein, these terms represent the percentage by weight of an ingredient in the recipe of the dosage unit.

“Infection” or Infestation”, as used herein, unless otherwise indicated, refers to the state or condition of having parasites on (ectoparasites) or in the body (endoparasites).

“Macrocyclic lactone” as used herein, designates a veterinary compound in the avermectin family of compounds including, for example, ivermectin, abamectin doramectin, eprinomectin, selamectin, and the like; and also includes the milbemycin family of compounds including, for example, moxidectin, milbemycin, milbemycin oxime, and the like. A preferred macrocyclic lactone of the composition of the invention is moxidectin. The preferred macrocyclic lactone is a stabilized macrocyclic lactone. The preferred stabilized macrocyclic lactone is moxidectin.

“Palatable”, as used herein, unless otherwise indicated, refers to a pleasant, acceptable, or agreeable taste. This results in a voluntary ingestion by animals either when offered by itself or at least when mixed into the animal's feed.

For use in the invention a ‘parasite’ is any noxious organism that negatively affects the health, well-being, or economic production level of a host animal. Parasites can be endo- or ectoparasites and may be attached in or on a host animal, and in a temporary or a stationary manner.

“Parasite(s)”, as used herein, unless otherwise indicated, refers to endoparasites and ectoparasites. Endoparasites are parasites that live within the body of its host and include helminths (e.g., trematodes, cestodes, and nematodes) and protozoa; but more particularly nematodes including gastrointestinal nematodes, lungworms, and heartworms. Ectoparasites are organisms of the Arthropoda phylum (e.g., arachnids and insects) which feed through or upon the skin of its host. Preferred arachnids are of the order Acarina, e.g., ticks and mites. Preferred insects are midges, lice (sucking and chewing), fleas, mosquitos, and biting flies (stable fly, horn fly, sand fly, blow fly, horse fly, and the like). Preferred compositions of the present invention can be used for the treatment of ectoparasites and endoparasites, i.e., treatment of a parasitic infection or infestation, including fleas, ticks, mites, lice, GI nematodes, and heartworm. Parasite(s) also encompasses the different life stages of the ectoparasite and endoparasite, including eggs, pupae, and larvae which feed on or in the body.

The ‘treatment’, and similar terms such as ‘treating’ or ‘treat’ as used herein, refer to the administration of an effective amount of the active drug substances as described for use in the invention to an animal which has an infestation—of more or less severity—with parasites of one or more species.

What constitutes an ‘effective amount’ for use in the invention, is the amount, therapeutic dose, or quantity of an isoxazoline as described herein, that is required for the complete eradication of the parasites infesting such animal, or for at least a significant reduction of the parasites infesting an animal. Alternatively, this may refer to an amount, dose, or quantity that can effectively control and/or reduce presence of parasites in an animal's housing or its surroundings, e.g. the house, building, farm, fields, etc.

‘Prevention’ or ‘prophylaxis’ means that a new infestation of the animal with parasites is prevented by killing adult parasites and any developmental or larval stage that is able to infest the host, before infestation of the host or directly after infestation of the protected host, and/or to prevent or reduce the development of new generations of parasites, in whole or in part.

“Stabilizing component” and “stabilizer”, as used herein, unless otherwise indicated, refers to an excipient or a combination of excipients (non-active ingredients) that have been found to provide chemical and/or physical stability, to one or more of the active agents, i.e., the macrocyclic lactones, veterinary acceptable isoxazolines of Formula (I), and/or pyrantel, especially pyrantel pamoate, that when combined together is more stable with the stabilizer(s) than without. For example, the stabilizing component stabilizes the macrocyclic lactone, i.e. prevents degradation, particularly, in case of moxidectin; resulting in stabilized moxidectin.

“Stable”, as used herein, unless otherwise indicated, refers to overall appearance, water content, assay, antioxidant (BHT) content, degradation products, dissolution, hardness, friability, photostability, and microbiological quality of the tablet. This can be measured in accordance with VICH Guidelines GL4 and GL5 under long-term and accelerated stability temperature and humidity conditions. Especially important is the diminishing of the appearance or growth of degradation products of active ingredients, especially of the macrocyclic lactone, especially moxidectin.

### DETAILED DESCRIPTION

The present invention provides a palatable veterinary chewable dosage form comprising an isoxazoline compound of Formula (I), pyrantel pamoate and a stabilized macrocyclic lactone compound selected from moxidectin and milbemycin oxime and a carrier comprising at least one flavor and a stabilizing component comprising an inorganic alkalizer, such as magnesium carbonate, porous silica or mixtures thereof. In a preferred embodiment such dosage form comprises a poloxamer

As is evident from the description and from the examples provided hereinafter, the inventors have devised a way to significantly improve the treatment of animals with a combination of active ingredients—an isoxazoline compound of Formula (I), pyrantel pamoate and a macrocyclic lactone by administration via a palatable chewable dosage form—in the form of a compressed tablet. This is because the incorporation of a stabilizing component provides protection to the macrocyclic lactone and the other active ingredients from degradation.

Therefore, in a further aspect the invention relates to the use of stabilizing component(s) as described in this application for the protection of a macrocyclic lactone compounds from degradation.

Additionally, it has been found that the veterinary chewable dosage form as compressed tablet is very palatable and voluntarily accepted by the animal.

It has been surprisingly found that the chewable veterinary dosage form is very palatable, resulting in almost complete voluntary uptake in dogs. This is unexpected because the tested tablet included only one flavor. In most of the prior art palatable composition, many flavors or palatability enhancers were required to see similar or even lower palatability results.

For use in the invention, the ‘isoxazoline’ is the following compound of the Formula (I)

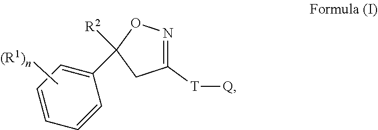

wherein
R¹ is halogen, CF₃, OCF₃, CN,
n is an integer from 0 up to and including 3, preferably 1, 2 or 3,
R² is C₁-C₃-haloalkyl, preferably CF₃ or CF₂Cl,
T is a 5 to 12 membered mono or bicyclic ring system, which is optionally substituted by one or more radicals Y,
Y is methyl, halomethyl, halogen, CN, NO₂, NH₂—C═S, or two adjacent radicals Y form together a chain, especially a three or four-membered chain;
Q is X—NR³R⁴, NR⁵—NR⁶—X—R³, X—R³ or a 5-membered N-heteroaryl ring, which is optionally substituted by one or more radicals;
X is CH₂, CH(CH₃), CH(CN), CO, CS,
R³ is hydrogen, methyl, haloethyl, halopropyl, halobutyl, methoxymethyl, methoxyethyl, halomethoxymethyl, ethoxymethyl, haloethoxymethyl, propoxymethyl, ethylaminocarbonylmethyl, ethylaminocarbonylethyl, dimethoxyethyl, propynylaminocarbonyl-methyl, N-phenyl-N-methyl-amino, haloethylaminocarbonylmethyl, haloethylaminocarbonylethyl, tetrahydrofuryl, methylaminocarbonylmethyl, (N,N-dimethylamino)-carbonylmethyl, propylaminocarbonylmethyl, cyclopropylaminocarbonylmethyl, propenylaminocarbonylmethyl, halo-ethylaminocarbonylcyclopropyl, alkylsulfanyl, alkylsufinalkyl, alkylsulfonalkyl, cycloalkyl

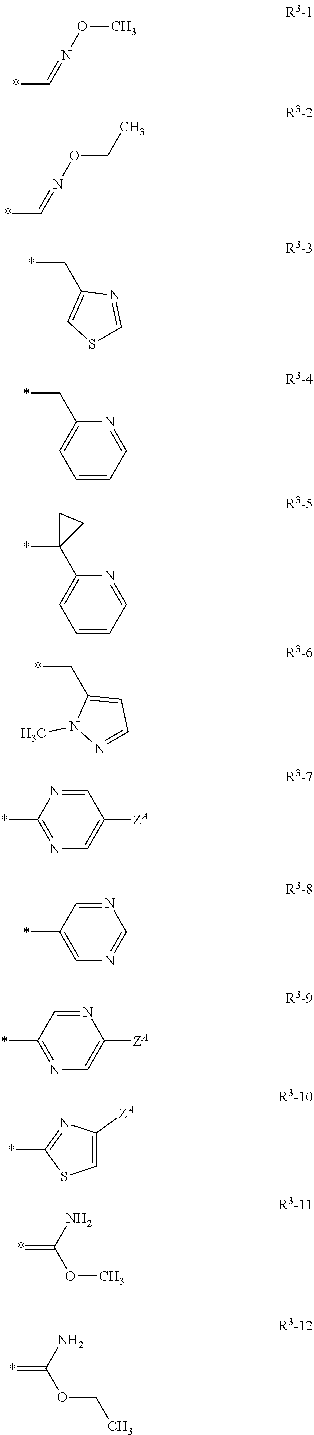

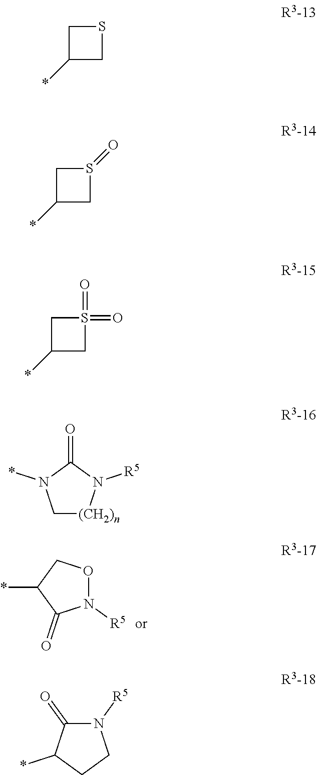

wherein Z^{A} is hydrogen, halogen, cyano, halomethyl, preferably CF₃;
R⁴ is hydrogen, ethyl, methoxymethyl, halomethoxymethyl, ethoxymethyl, haloethoxymethyl, propoxymethyl, methylcarbonyl, ethylcarbonyl, propylcarbonyl, cyclopropylcarbonyl, methoxycarbonyl, methoxymethylcarbonyl, aminocarbonyl, ethylaminocarbonylmethyl, ethylaminocarbonylethyl, dimethoxyethyl, propynylaminocarbonylmethyl, haloethylaminocarbonylmethyl, cyanomethylaminocarbonylmethyl, or haloethyl-aminocarbonylethyl;
R⁵ is hydrogen, alkyl or haloalkyl;
R⁶ is hydrogen, alkyl or haloalkyl;
or R³ and R⁴ together form a substituent selected from the group consisting of:

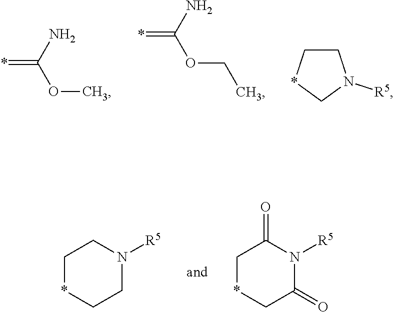

or a salt or solvate thereof.

In a preferred embodiment of the invention and/or embodiments thereof T is selected from

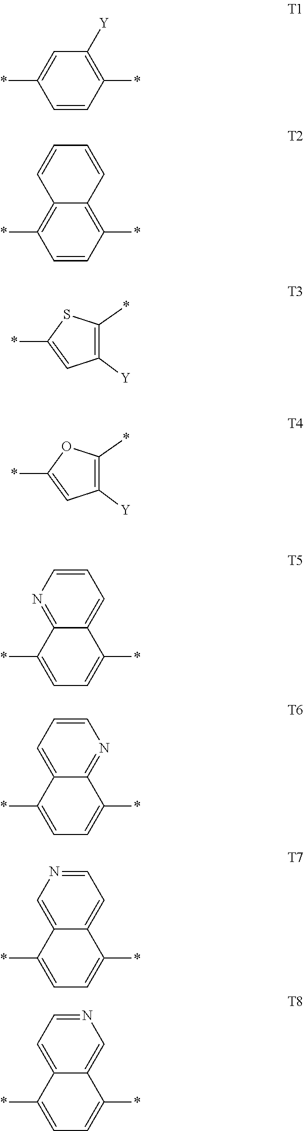

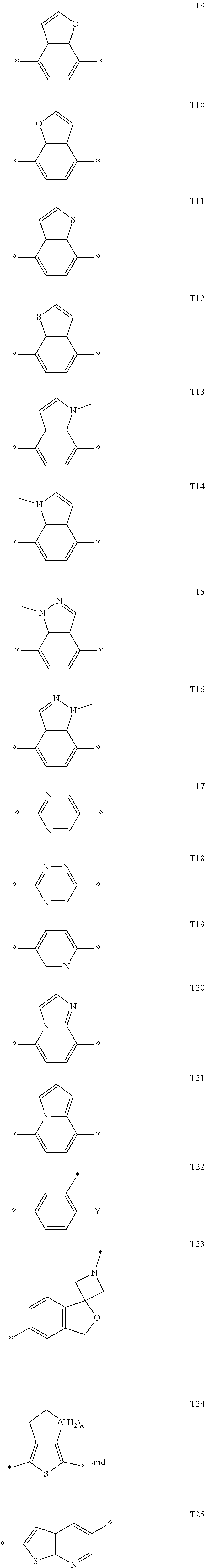

wherein in T-1, T-3 and T-4, the radical Y is preferably hydrogen, halogen, methyl, halomethyl, ethyl or haloethyl.

In a preferred embodiment of the invention and/or embodiments thereof Q in Formula (I) is selected from

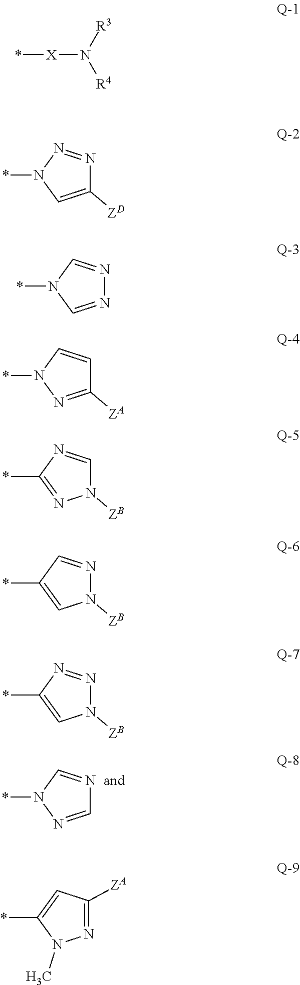

wherein R³, R⁴, X and Z^{A} are as defined above and

Z^{B} is

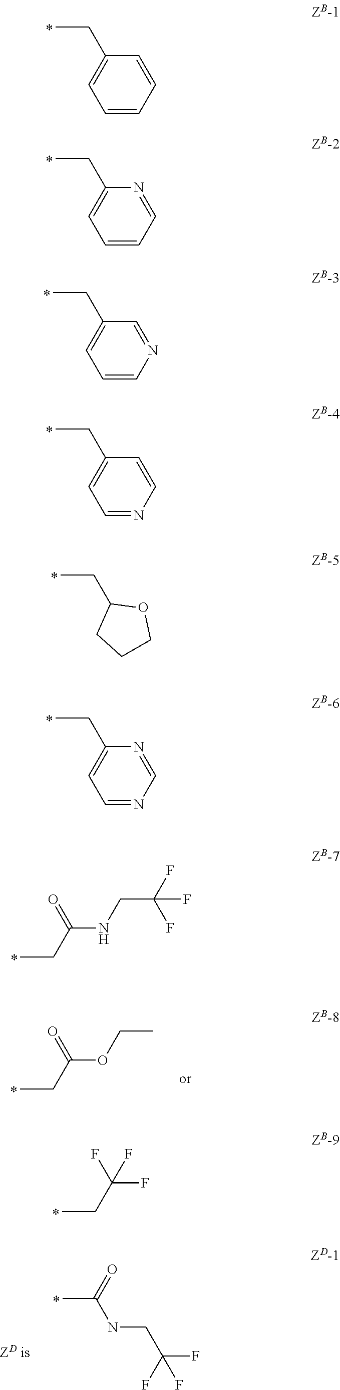

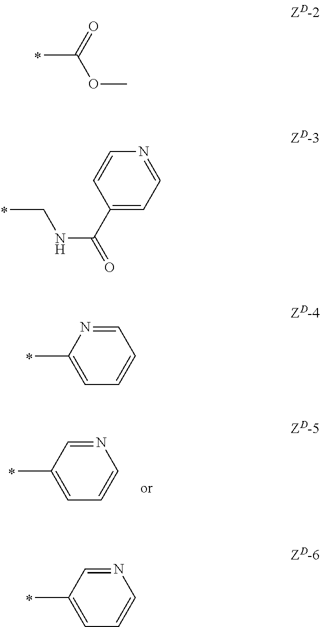

Preferred compounds of Formula (I) are listed in Table 1:

TABLE 1

| (R¹)ₙ | R² | R³ | R⁴ | T | Y | Q | Z | X |
|---|---|---|---|---|---|---|---|---|
| 3-Cl, 5Cl | CF₃ | CH₂CF₃ | H | T-2 | — | Q-1 | — | C(O) |
| 3-Cl, 5Cl | CF₃ | CH₂CH₃ | H | T-2 | — | Q-1 | — | C(O) |
| 3-Cl, 5Cl | CF₃ | CH₂CH₂OCH₃ | H | T-2 | — | Q-1 | — | C(O) |
| 3-Cl, 5Cl | CF₃ | CH₂C(O)NHCH₂CF₃ | H | T-2 | — | Q-1 | — | C(O) |
| 3-Cl, 5Cl | CF₃ | CH₂C(O)NHCH₂CH₃ | H | T-2 | — | Q-1 | — | C(O) |
| 3-CF₃, 5-CF₃ | CF₃ | CH₂C(O)NHCH₂CF₃ | H | T-2 | — | Q-1 | — | C(O) |
| 3-CF₃, 5-CF₃ | CF₃ | CH₂C(O)NHCH₂CH₃ | H | T-2 | — | Q-1 | — | C(O) |
| 3-CF₃, 5-Cl | CF₃ | CH₂C(O)NHCH₂CF₃ | H | T-2 | — | Q-1 | — | C(O) |
| 3-CF₃, 5-Cl | CF₃ | CH₂C(O)NHCH₂CH₃ | H | T-2 | — | Q-1 | — | C(O) |
| 3-Cl, 5Cl | CF₃ | — | | T-2 | — | Q-6 | Z^{B}-7 | |
| 3-Cl, 5Cl | CF₃ | — | — | T-2 | — | Q-7 | Z^{B}-7 | |
| 3-Cl, 5Cl | CF₃ | — | — | T-2 | — | Q-5 | Z^{B}-7 | |
| 3-Cl, 5Cl | CF₃ | — | — | T-2 | — | Q-2 | Z^{D}-1 | |
| 3-Cl, 5Cl | CF₃ | CH₂C(O)NHCH₂CF₃ | H | T-3 | CH₃ | Q-1 | — | C(O) |
| 3-Cl, 5Cl | CF₃ | CH₂C(O)NHCH₂CC | H | T-3 | CH₃ | Q-1 | — | C(O) |
| 3-Cl, 5Cl | CF₃ | CH₂C(O)NHCH₂CN | H | T-3 | CH₃ | Q-1 | — | C(O) |
| 3-Cl, 5Cl | CF₃ | CH₂C(O)NHCH₂CH₃ | H | T-3 | CH₃ | Q-1 | — | C(O) |
| 3-CF₃, 5-CF₃ | CF₃ | CH₂C(O)NHCH₂CF₃ | H | T-3 | CH₃ | Q-1 | — | C(O) |
| 3-CF₃, 5-CF₃ | CF₃ | CH₂C(O)NHCH₂CH₃ | H | T-3 | CH₃ | Q-1 | — | C(O) |
| 3-Cl, 4-Cl, 5-Cl | CF₃ | CH₂C(O)NHCH₂CF₃ | H | T-3 | CH₃ | Q-1 | — | C(O) |
| 3-Cl, 4-Cl, 5-Cl | CF₃ | CH₂C(O)NHCH₂CH₃ | H | T-3 | CH₃ | Q-1 | — | C(O) |
| 3-Cl, 4-F, 5-Cl | CF₃ | CH₂C(O)NHCH₂CF₃ | H | T-3 | CH₃ | Q-1 | — | C(O) |
| 3-Cl, 4-F, 5-Cl | CF₃ | CH₂C(O)NHCH₂CH₃ | H | T-3 | CH₃ | Q-1 | — | C(O) |
| 3-Cl, 5-Cl | CF₃ | CH₂C(O)NHCH₂CF₃ | H | T-20 | — | Q-1 | — | C(O) |
| 3-Cl, 5-Cl | CF₃ | CH₂C(O)NHCH₂CH₃ | H | T-20 | — | Q-1 | — | C(O) |
| 3-CF₃, 5-CF₃ | CF₃ | CH₂C(O)NHCH₂CF₃ | CH₃ | T-20 | — | Q-1 | — | C(O) |
| 3-CF₃, 5-CF₃ | CF₃ | CH₂C(O)NHCH₂CH₃ | CH₃ | T-20 | — | Q-1 | — | C(O) |
| 3-CF₃, 5-CF₃ | CF₃ | CH₂C(O)NHCH₂CF₃ | H | T-20 | — | Q-1 | — | C(O) |
| 3-CF₃, 5-CF₃ | CF₃ | CH₂C(O)NHCH₂CH₃ | H | T-20 | — | Q-1 | — | C(O) |
| 3-CF₃, 5-CF₃ | CF₃ | CH₂C(O)NHCH₂CF₃ | H | T-21 | — | Q-1 | — | C(O) |
| 3-CF₃, 5-CF₃ | CF₃ | CH₂C(O)NHCH₂CH₃ | H | T-21 | — | Q-1 | — | C(O) |
| 3-Cl, 5-Cl | CF₃ | CH₂C(O)NHCH₂CF₃ | H | T-21 | — | Q-1 | — | C(O) |
| 3-Cl, 5-Cl | CF₃ | CH₂C(O)NHCH₂CH₃ | H | T-21 | — | Q-1 | — | C(O) |
| 3-Cl, 5-Cl | CF₃ | CH₂CH₂SCH₃ | H | T-21 | — | Q-1 | — | C(O) |
| 3-Cl, 4-Cl, 5-Cl | CF₃ | C(O)CH₃ | H | T-22 | F | Q-1 | — | CH₂ |
| 3-Cl, 4-Cl, 5-Cl | CF₃ | C(O)CH(CH₃)₂ | H | T-22 | F | Q-1 | — | CH₂ |
| 3-Cl, 4-Cl, 5-Cl | CF₃ | C(O)-cyclo-propyl | H | T-22 | F | Q-1 | — | CH₂ |
| 3-Cl, 4-F, 5-Cl | CF₃ | C(O)CH₃ | H | T-22 | F | Q-1 | — | CH₂ |
| 3-Cl, 4-Cl, 5-Cl | CF₃ | C(O)CH₂CH₃ | H | T-22 | F | Q-1 | — | CH₂ |
| 3-Cl, 4-F, 5-Cl | CF₃ | C(O)CH₃ | H | T-22 | Cl | Q-1 | — | CH₂ |
| 3-Cl, 5-Cl | CF₃ | CH₂C(O)NHCH₂CF₃ | H | T-1 | CH₃ | Q-1 | — | C(O) |
| 3-Cl, 5-Cl | CF₃ | CH₂C(O)NHCH₂CH₃ | H | T-1 | CH₃ | Q-1 | — | C(O) |
| 3-Cl, 5-Cl | CF₃ | R³-1 (Z) | H | T-1 | CH₃ | Q-1 | — | C(O) |
| 3-Cl, 5-Cl | CF₃ | R³-1 (E) | H | T-1 | CH₃ | Q-1 | — | C(O) |

More preferred compounds of Formula (I) are listed in Table 2.

TABLE 2

| (R¹)ₙ | R² | R³ | R⁴ | T | Y | Q | Z | X |
|---|---|---|---|---|---|---|---|---|
| 3-Cl, 5Cl | CF₃ | CH₂CF₃ | H | T-2 | — | Q-1 | — | C(O) |
| 3-Cl, 5Cl | CF₃ | CH₂CH₃ | H | T-2 | — | Q-1 | — | C(O) |
| 3-Cl, 5Cl | CF₃ | CH₂CH₂OCH₃ | H | T-2 | — | Q-1 | — | C(O) |
| 3-Cl, 5Cl | CF₃ | CH₂C(O)NHCH₂CF₃ | H | T-2 | — | Q-1 | — | C(O) |
| 3-CF₃, 5-CF₃ | CF₃ | CH₂C(O)NHCH₂CF₃ | H | T-2 | — | Q-1 | — | C(O) |
| 3-CF₃, 5-Cl | CF₃ | CH₂C(O)NHCH₂CF₃ | H | T-2 | — | Q-1 | — | C(O) |
| 3-Cl, 5Cl | CF₃ | — | | T-2 | — | Q-6 | Z^{B}-7 | |
| 3-Cl, 5Cl | CF₃ | — | — | T-2 | — | Q-7 | Z^{B}-7 | |
| 3-Cl, 5Cl | CF₃ | — | — | T-2 | — | Q-5 | Z^{B}-7 | |
| 3-Cl, 5Cl | CF₃ | — | — | T-2 | — | Q-2 | Z^{D}-1 | |
| 3-Cl, 5Cl | CF₃ | CH₂C(O)NHCH₂CF₃ | H | T-3 | CH₃ | Q-1 | — | C(O) |
| 3-Cl, 5Cl | CF₃ | CH₂C(O)NHCH₂CC | H | T-3 | CH₃ | Q-1 | — | C(O) |
| 3-Cl, 5Cl | CF₃ | CH₂C(O)NHCH₂CN | H | T-3 | CH₃ | Q-1 | — | C(O) |
| 3-CF₃, 5-CF₃ | CF₃ | CH₂C(O)NHCH₂CF₃ | H | T-3 | CH₃ | Q-1 | — | C(O) |
| 3-Cl, 4-Cl, 5-Cl | CF₃ | CH₂C(O)NHCH₂CF₃ | H | T-3 | CH₃ | Q-1 | — | C(O) |
| 3-Cl, 4-F, 5-Cl | CF₃ | CH₂C(O)NHCH₂CF₃ | H | T-3 | CH₃ | Q-1 | — | C(O) |
| 3-Cl, 5-Cl | CF₃ | CH₂C(O)NHCH₂CF₃ | H | T-20 | — | Q-1 | — | C(O) |
| 3-CF₃, 5-CF₃ | CF₃ | CH₂C(O)NHCH₂CF₃ | CH₃ | T-20 | — | Q-1 | — | C(O) |
| 3-CF₃, 5-CF₃ | CF₃ | CH₂C(O)NHCH₂CF₃ | H | T-20 | — | Q-1 | — | C(O) |
| 3-CF₃, 5-CF₃ | CF₃ | CH₂C(O)NHCH₂CF₃ | H | T-21 | — | Q-1 | — | C(O) |
| 3-Cl, 5-Cl | CF₃ | CH₂C(O)NHCH₂CF₃ | H | T-21 | — | Q-1 | — | C(O) |
| 3-Cl, 5-Cl | CF₃ | CH₂CH₂SCH₃ | H | T-21 | — | Q-1 | — | C(O) |
| 3-Cl, 4-Cl, 5-Cl | CF₃ | C(O)CH₃ | H | T-22 | F | Q-1 | — | CH₂ |
| 3-Cl, 4-Cl, 5-Cl | CF₃ | C(O)CH(CH₃)₂ | H | T-22 | F | Q-1 | — | CH₂ |
| 3-Cl, 4-Cl, 5-Cl | CF₃ | C(O)-cyclo-propyl | H | T-22 | F | Q-1 | — | CH₂ |
| 3-Cl, 4-F, 5-Cl | CF₃ | C(O)CH₃ | H | T-22 | F | Q-1 | — | CH₂ |
| 3-Cl, 4-Cl, 5-Cl | CF₃ | C(O)CH₂CH₃ | H | T-22 | F | Q-1 | — | CH₂ |
| 3-Cl, 4-F, 5-Cl | CF₃ | C(O)CH₃ | H | T-22 | Cl | Q-1 | — | CH₂ |
| 3-Cl, 5-Cl | CF₃ | CH₂C(O)NHCH₂CF₃ | H | T-1 | CH₃ | Q-1 | — | C(O) |
| 3-Cl, 5-Cl | CF₃ | R³-1 (Z) | H | T-1 | CH₃ | Q-1 | — | C(O) |
| 3-Cl, 5-Cl | CF₃ | R³-1 (E) | H | T-1 | CH₃ | Q-1 | — | C(O) |

In a particularly preferred embodiment of the invention and/or embodiments thereof the isoxazoline compound is represented by Formula (II)

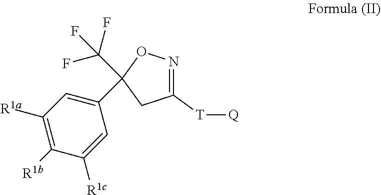

wherein
R^{1a}, R^{1b}, R^{1c} are independently from each other hydrogen, Cl or CF₃. Preferably R^{1a} and R^{1c} are Cl or CF₃ and R^{1b} is hydrogen,
T is

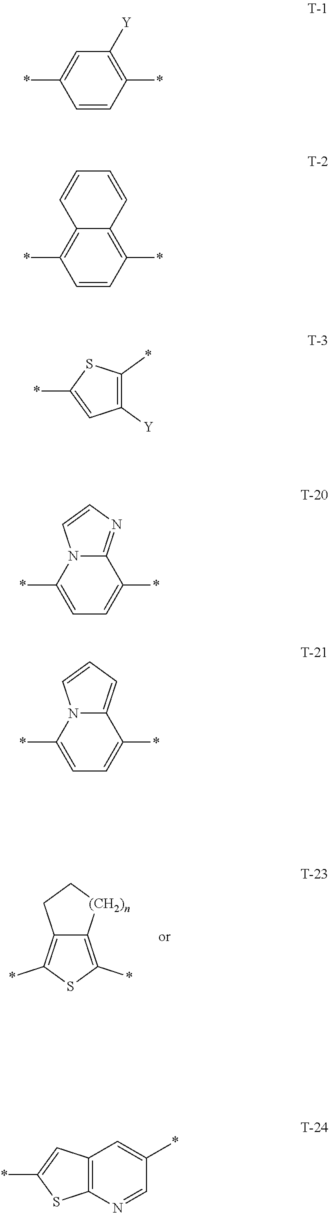

wherein Y is methyl, bromine, Cl, F, CN or C(S)NH₂ and
Q is as described above.

In another preferred embodiment of the invention and/or embodiments thereof R³ is H and R⁴ is —CH₂—C(O)—NH—CH₂—CF₃, —CH₂—C(O)—NH—CH₂—CH₃, —CH₂—CH₂—CF₃ or —CH₂—CF₃.

In another preferred embodiment of the invention and/or embodiments thereof the isoxazoline compound is selected from fluralaner, afoxolaner, esafoxolaner, sarolaner, lotilaner and tigolaner.

In one preferred embodiment of the invention and/or embodiments thereof the isoxazoline is 4-[5-(3,5-dichlorophenyl)-5-trifluoromethyl-4,5-dihydroisoxazol-3-yl]-2-methyl-N-[(2,2,2-trifluoro-ethylcarbamoyl)-methyl]-benzamide (CAS RN 864731-61-3). This compound is also known as fluralaner.

In one preferred embodiment of the invention and/or embodiments thereof the isoxazoline is 4-[5-[3-chloro-5-(trifluoromethyl)phenyl]-4,5-dihydro-5-(trifluoromethyl)-3-isoxazolyl]-N-[2-oxo-2-[(2,2,2-trifluoroethyl)amino]ethyl]-1-naphthalene-carboxamide (CAS RN1093861-60-9). This compound is also known as a 4-[5-(5-chloro-α,α,α-trifluoro-m-tolyl)-4,5-dihydro-5-(trifluoromethyl)-1,2-oxazol-3yl]-N-[2-oxo-2-[(2,2,2-trifluoroethylamino]ethyl]naphthalene-1- or afoxolaner. Afoxolaner is for example disclosed in WO 2007/079162. In one embodiment the isoxazoline compound is Esafoxolaner.

In one preferred embodiment of the invention and/or embodiments thereof the isoxazoline is 1-(5′-(5-(3,5-dichloro-4-fluorophenyl)-5-(trifluoromethyl)-4,5-dihydroisoxazol-3-yl)-3′H-spiro[azetidine-3,1′-isobenzofuran]-1-yl)-2-(methylsulfonyl)ethan-1-one, preferably 1-(5′-((5S)-(5-(3,5-dichloro-4-fluorophenyl)-5-(trifluoromethyl)-4,5-dihydroisoxazol-3-yl)-3′H-spiro[azetidine-3,1′-isobenzofuran]-1-yl)-2-(methylsulfonyl)ethan-1-one (CAS RN: 1398609-39-6). This compound is known as sarolaner.

In one preferred embodiment of the invention and/or embodiments thereof the isoxazoline is 3-methyl-N-(2-oxo-2-((2,2,2-trifluoroethyl)amino)ethyl)-5-[5-(3,4,5-trichlorophenyl)-5-(trifluoromethyl)-4,5-dihydroisoxazol-3-yl]thiophene-2-carboxamide, preferably methyl-N-(2-oxo-2-((2,2,2-trifluoroethyl)amino)ethyl)-5-[(5S)-5(3,4,5-trichlorophenyl)-5-(trifluoromethyl)-4,5-dihydroisoxazol-3-yl]thiophene-2-carboxamide (CAS RN: 1369852-71-0). This compound is known as lotilaner.

In one preferred embodiment of the invention and/or embodiments thereof a compound used as alternative to the isoxazoline compound is 2-chloro-N-(1-cyanocyclopropyl)-5-[1-[2-methyl-5-(1,1,2,2,2-pentafluoroethyl)-4-(trifluoromethyl)pyrazol-3-yl]pyrazol-4-yl]benzamide (CAS RN 1621436) This compound is known as tigolaner.

In one preferred embodiment of the invention and/or embodiments thereof the isoxazoline is 4H-Cyclopenta[c]thiophene-1-carboxamide, 3-[(5S)-5-(3,5-dichloro-4-fluorophenyl)-4,5-dihydro-5-(trifluoromethyl)-3-isoxazolyl]-N-[2-[(2,2-difluoroethyl)amino]-2-oxoethyl]-5,6-dihydro-(CAS 1414642-93-5). This compound is known as mivorilaner.

In one preferred embodiment of the invention and/or embodiments thereof the isoxazoline is In another embodiment the compound of Formula (I) is (Z)-4-[5-(3,5-dichlorophenyl)-5-trifluoromethyl-4,5-dihydroisoxazol-3-yl]-N-[(methoxyimino)methyl]-2-methylbenzamide (CAS RN 928789-76-8).

In one preferred embodiment of the invention and/or embodiments thereof the isoxazoline is 4-[5-(3,5-dichlorophenyl)-5-(trifluoromethyl)-4H-isoxazol-3-yl]-2-methyl-N-(thietan-3-yl)benzamide (CAS RN 1164267-94-0) that was disclosed in WO 2009/0080250.

In one preferred embodiment of the invention and/or embodiments thereof the isoxazoline is 5-[5-(3,5-dichlorophenyl)-4,5-dihydro-5-(trifluoromethyl)-3-isoxazolyl]-3-methyl-N-[2-oxo-2-[(2,2,2-trifluoroethyl)amino]ethyl]-2-thiophenecarboxamide (CAS RN 1231754-09-8) that was disclosed in WO 2010/070068.

Especially preferred is fluralaner (corresponding to 4-[5-(3,5-dichlorophenyl)-5-trifluoromethyl-4,5-dihydroisoxazol-3-yl]-2-methyl-N-[(2,2,2-trifluoro-ethylcarbamoyl)-methyl]-benzamide) as systemic insecticide and/or acaricide (a) which is represented by Formula (III)

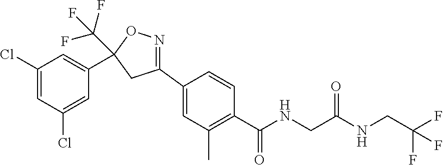 [SMILES: Cc1cc(C2=NOC(c3cc(Cl)cc(Cl)c3)(C(F)(F)F)C2)ccc1C(=O)NCC(=O)NCC(F)(F)F]

The isoxazoline compounds may exist in various isomeric forms. A reference to an isoxazoline compound always includes all possible isomeric forms of such a compound. Unless otherwise stated, a compound structure that does not indicate a particular conformation is intended to encompass compositions of all the possible conformational isomers of the compound, as well as compositions comprising fewer than all the possible conformational isomers. In some embodiments, the compound is a chiral compound. In some embodiments, the compound is a non-chiral compound.

Isoxazoline compounds of Formula (I) can be prepared according to one or other of the processes described e.g. in patent applications US 2007/0066617, WO 2007/079162, WO 2009/002809, WO 2009/080250, WO 2010/070068, WO 2010/079077, WO 2011/075591 and WO 2011/124998 or any other process coming within the competence of a person skilled in the art who is an expert in chemical synthesis.

For the chemical preparation, a person skilled in the art is regarded as having at her/his disposal, inter alia, the entire contents of “Chemical Abstracts” and of the documents cited therein.

In one preferred embodiment of the invention and/or embodiments thereof the amount of the isoxazoline compound comprised in the palatable chewable veterinary dosage form may be in the range of from about 1 to about 30 weight %. In an alternative embodiment the amount of such compound may be in the range of from about 2 to about 20 weight %. The preferred range is from about 5 to about 18 weight %, in particular from about 10 to about 15 weight %.

Pyrantel pamoate is a known anthelmintic component. Pyrantel pamoate is present in the chewable veterinary dosage form about 5 weight % to about 30 weight %, preferably about 10 weight % to about 25 weight %, more preferably about 15 weight % to about 20 weight %.

Physiologically active macrocyclic lactones (also referred to as or macrolides or macrocyclic lactones-ML) are organic molecules comprising a ring structure, wherein said molecules include a lactone group. Such a lactone group can also be considered as intramolecular carboxylic acid ester group. Macrocyclic lactones are often found in metabolic products in bacteria and fungi. Furthermore, in one embodiment, the palatable chewable veterinary dosage forms of the invention may comprise a combination of two or more macrocyclic lactone active agents.

For the avoidance of doubt, the term “macrocyclic lactone” as used herein includes both naturally occurring and synthetic or semi-synthetic macrocyclic lactones, especially parasiticidal avermectin and milbemycin compounds.

The macrocyclic lactones that may be used in the palatable chewable veterinary dosage form of the invention include, but are not limited to, naturally produced avermectins (e.g. including the components designated as A1a, A1b, A2a, A1b, B1a, B1b, B2a and B2b) and milbemycin compounds, semisynthetic avermectins and milbemycins, avermectin monosaccharide compounds and avermectin aglycone compounds. Examples of macrocyclic lactone compounds that may be used in the compositions include, but are not limited to, abamectin, dimadectin, doramectin, emamectin, eprinomectin, ivermectin, latidectin, lepimectin, selamectin, ML-1,694,554 and milbemycins including, but not limited to, milbemectin, milbemycin D, milbemycin A3, milbemycin A4, milbemycin oxime, moxidectin and nemadectin. Also included are the 5-oxo and 5-oxime derivatives of said avermectins and milbemycins.

The macrocyclic lactone compounds are known in the art and can easily be obtained commercially or through synthesis techniques known in the art. Reference is made to the widely available technical and commercial literature.

In one preferred embodiment of the invention and/or embodiments thereof the one or more physiologically active macrocyclic lactone(s) (f1) is selected from abamectin, dimadectin, doramectin, emamectin, eprinomectin, ivermectin, latidectin, lepimectin, selamectin, ML-1,694,554 and milbemycins including, but not limited to, milbemectin, milbemycin D, milbemycin A3, milbemycin A4, milbemycin oxime, moxidectin, nemadectin and mixtures thereof.

In one preferred embodiment of the invention and/or embodiments thereof the one or more physiologically active macrocyclic lactone(s) is selected from ivermectin, abamectin, milbemycin oxime, moxidectin, doramectin, selamectin, eprinomectin, emamectin and mixtures thereof. More preferred as physiologically active macrocyclic lactone(s) is milbemycin oxime or moxidectin or alternatively ivermectin. Most preferred is moxidectin.

In one preferred embodiment of the invention and/or embodiments thereof the amount of the macrocyclic lactone(s) may be in the range of from about 0.001 to about 10 weight %, of the composition, depending on the effective concentration that varies between the different macrocyclic lactone compounds.

In one preferred embodiment of the invention and/or embodiments thereof the one or more highly active physiologically active macrocyclic lactone(s) such as moxidectin comprised in the palatable chewable veterinary dosage form may be in the range of from about 0.0075 to about 0.075 weight %. In an alternative embodiment the amount of moxidectin may be in the range of from about 0.01 to about 0.07 weight %. The preferred range is from about 0.0125 to about 0.065 weight %.

In case that the physiologically active macrocyclic lactone is milbemycin oxime, the amount thereof may be in the range from 0.5 to 20 weight %, preferably about 1 weight %, about 2 weight %, about 3 weight %, about 4 weight, or about 5 weight % of the aggregate.

In one preferred embodiment of the invention and/or embodiments thereof the macrocyclic lactone(s) is ivermectin comprised in the palatable chewable veterinary dosage form may be in the range of from 0.0075 to 0.075 weight %, preferably about 0.015 weight %, about 0.0225 weight %, about 0.03 weight %, about 0.0375 weight %.

Other pharmaceutical agents, such as vitamins, mineral supplements, which are known in the veterinary art are also contemplated to be included in the palatable chewable veterinary dosage form according to the invention.

Manufacture of the palatable chewable veterinary dosage forms of the invention in the form of a hard compressed tablet or alternatively as a soft chewable composition may involve a process of preparing several separate granulations and combining them into the palatable chewable veterinary dosage form, e.g., the manufacture of granules containing the moxidectin active ingredient and a second granulation containing the isoxazoline, e.g. fluralaner, and pyrantel active ingredient, e.g. pyrantel pamoate.

These granulations can be e.g. then blended with the extra-granular materials and compressed into the final palatable chewable veterinary dosage form in the form of a hard compressed tablet. Alternatively, the granules (or granulations) are included in a soft chewable veterinary composition that is formed by molding, e.g., rotary molding or extrusion.

In the palatable chewable veterinary dosage form of the invention the macrocyclic lactone compound, especially moxidectin is stabilized with a stabilizing component that comprises an inorganic alkalizer, porous silica or mixtures thereof. In one preferred embodiment the stabilizing component includes a poloxamer.

Components of the stabilizing component can be present in any of these components of the palatable chewable veterinary dosage form, in any of the granules (or granulations) or in extra-granular material.

Moxidectin is prone to hydrolytic and oxidative degradation. Stability issues can be observed due to its interaction with flavor and other inactive ingredients used in a tablet formulation.

The stabilizing component addresses these challenges and avoids the formation of moxidectin degradation products.

The amount of the highly potent macrocyclic lactone moxidectin (in the palatable chewable veterinary dosage form of the invention is very low compared to the other active ingredients present. Degradation can therefore easily lead to a combination product with sub-therapeutic levels of moxidectin. Moxidectin degradants are known and include the acid catalyzed degradants 23-Z-moxidectin, 23-keo nemadectin and 23-keto-alpha moxidectin as well as base catalyzed degradants delta-2-moxidectin and 2-epimer moxidectin.

In addition to stability, the low amount of moxidectin in the palatable chewable veterinary dosage form can also lead to content uniformity issues of the drug product. To overcome moxidectin degradation and improve content uniformity and to ensure a stable homogenous composition, moxidectin was stabilized with a stabilizing component to address these challenges.

Besides stability, the stabilizing approach also aids in the homogeneous distribution of moxidectin in the granulation as well as in the final tablet composition to ensure content uniformity. An additional hydrophilic polymer, especially non cellulosic polymer and antioxidant can be added to the moxidectin granulation to provide added stability to ensure longer shelf-life of the final drug product.

In an alternative the hydrophilic polymer, especially non cellulosic polymer and antioxidant, when added to the moxidectin granulation, improve stability compared to when used alone.

In one embodiment the stabilizing component comprises at least one inorganic alkalizer, porous silica or mixtures thereof.

An inorganic alkalizer can be used to help stabilize the active ingredients in the chewable veterinary dosage form, in part (compositional granulations and common blend) or the tablet.

In one embodiment the inorganic alkalizer is incorporated into the moxidectin granulation.

Preferably the inorganic alkalizer is a magnesium carbonate. The most common magnesium carbonate forms are the anhydrous salt called magnesite (MgCO₃) and the di, tri, and pentahydrates known as barringtonite (MgCO3·2 H2O), nesquehonite (MgCO³·3 H₂O), and lansfordite (MgCO₃·5 H₂O), respectively. Some basic forms such as artinite (MgCO₃—Mg(OH)₂·3 H₂O), hydromagnesite (4 MgCO₃—Mg(OH)₂-4·H₂O), and dypingite (4 MgCO₃·Mg(OH)₂-5 H₂O) also occur as minerals. Preferably the magnesium carbonate is light magnesium carbonate.

Light Magnesium Carbonate is an inorganic compound with the chemical formula MgCO₃.

The difference between light and heavy magnesium carbonate is that the light magnesium carbonate consists of 4 water molecules whereas the heavy magnesium carbonate contains 5 water molecules. References to “light” and “heavy” magnesium carbonates actually refer to the magnesium hydroxy carbonates hydromagnesite and dypingite (respectively).

Magnesium carbonate can be included in any of the granulation components or in the extragranular material. In one embodiment part of the magnesium carbonate is included in the moxidectin granulation.

In a specific embodiment the magnesium carbonate is included in the dry mix that is used to prepare the moxidectin granulation.

In a specific embodiment part of the magnesium carbonate is included in the moxidectin solution and part in the dry mix that forms the moxidectin granulation. In another embodiment the magnesium carbonate is included in the moxidectin solution that is used to form the moxidectin granulation.

In one preferred embodiment of the invention and/or embodiments thereof the inorganic alkalizer comprised in the chewable veterinary dosage form may be in the range of from about 0.5 to about 5 weight %. The preferred range is from about 1.2 to about 1.8 weight %.

In an embodiment the amount of magnesium carbonate in the moxidectin granulation may be in the range of from about 1 to about 8 weight %, especially 6%.

Porous silica can be used to help stabilize the active ingredients in the chewable veterinary dosage form, in part (compositional granulations and common blend) or the tablet. In one embodiment the porous silica is incorporated into the moxidectin granulation.

In one embodiment the porous silica is magnesium aluminum silicate. Magnesium aluminum silicate is an off-white powder used in the pharmaceutical manufacturing process as an absorbent; anticaking agent; opacifying agent; slip modifier; and an aqueous viscosity increasing agent. It can be used in tablet making and in medication suspensions, and it is also used in the cosmetics industry. Neutral or alkaline grade of magnesium aluminometasilicate are known.

Neusilin® is a synthetic, amorphous form of magnesium aluminometasilicate. It is a multifunctional excipient that can be used in both direct compression and wet granulation of solid dosage forms. Especially preferred is Neusilin® US2 (CAS Number:12511-31-8) is e.g. available from Fuji Chemical Industry Co, Ltd.

In one preferred embodiment of the invention and/or embodiments thereof the porous silica comprised in the chewable veterinary dosage form may be in the range of from about 1 to about 15 weight %. In an alternative embodiment the amount of moxidectin may be in the range of from about 2 to about 10 weight %. The preferred range is from about 4 to about 8 weight %.

The stabilizing component preferably comprises a hydrophilic polymer. Hydrophilic polymers can be used to help stabilize the active ingredients in the chewable veterinary dosage form, in part (compositional granulations and common blend) or the tablet, i.e. the final blended and compressed composition. In one embodiment the hydrophilic polymer comprises a non-cellulosic polymer, preferably a Poloxamer. Poloxamers are nonionic triblock copolymers composed of a central hydrophobic chain of polyoxypropylene (poly (propylene oxide)) flanked by two hydrophilic chains of polyoxyethylene (poly(ethylene oxide)).

Poloxamers are also known by the trade names Pluronic and Kolliphor (pharma grade).

Because the lengths of the polymer blocks can be customized, many different poloxamers exist that have slightly different properties. For the generic term poloxamer, these copolymers are commonly named with the letter P (for poloxamer) followed by three digits: the first two digits multiplied by 100 give the approximate molecular mass of the polyoxypropylene core, and the last digit multiplied by 10 gives the percentage polyoxyethylene content (e.g. P407=poloxamer with a polyoxypropylene molecular mass of 4000 g/mol} and a 70% polyoxyethylene content).

Especially preferred is Poloxamer 188.

Poloxamer 188 is a nonionic block copolymer of ethylene oxide and propylene oxide as represented by the following structure, where the a and b blocks contain 80 and 27 units, respectively:

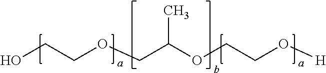

It has an average molecular weight of 7680-9510 g/mol. The surfactant properties of poloxamer 188 make the copolymer useful in cosmetic, pharmaceutical, and industrial applications. Its stabilizing role in the chewable dosage form of the current invention was surprising.

In one preferred embodiment of the invention and/or embodiments thereof the poloxamer comprised in the chewable veterinary dosage form may be in the range of from about 1 to about 15 weight %. In an alternative embodiment the amount of moxidectin may be in the range of from about 2 to about 10 weight %. The preferred range is from about 4 to about 10 weight %.

The inclusion of Poloxamer has been shown to have a very positive impact on diminishing the degradation of moxidectin during processing and on the stability during storage, especially under normal temperature conditions and therefore on the shelf life of the chewable veterinary dosage form.

Poloxamer is preferably included in the macrocyclic lactone, especially moxidectin granulation that is then combined with the one or more additional granulations and extragranular material into the chewable veterinary dosage form.

A preferred embodiment is a macrocyclic lactone, especially moxidectin granulation comprising stabilized moxidectin. In a preferred embodiment such moxidectin granulation comprises at least one poloxamer, especially poloxamer 188, preferably in combination with at least one other stabilizing component selected from BHT, magnesiummetasilicate, light magnesium carbonate and microcrystalline celluloses.

In one embodiment such moxidectin granulation is included in a flavored compressed tablet.

In an alternative embodiment the granulation with the macrocyclic lactone, especially moxidectin that has been stabilized according to the current invention is used in a process to incorporate moxidectin in the form of a moxidectin granulation in a soft chewable veterinary dosage form.

In another embodiment the hydrophilic polymer comprises a cellulosic polymer, especially HPMC. Hypromellose (INN), short for hydroxypropyl methylcellulose (HPMC), is a semisynthetic, inert, viscoelastic polymer used as eye drops, as well as an excipient and controlled-delivery component in oral medicaments, found in a variety of commercial products. HPMC has been used as an excipient in oral tablet and capsule formulations, where, depending on the grade, it functions as binder or controlled release agent.

In another embodiment the hydrophilic polymer comprises a combination of a cellulosic and non-cellulosic polymer, especially preferred a Poloxamer and HPMC, preferably Poloxamer 188 and HPMC.

Especially useful is HPMC of USP 2910 with 28-30% Methoxy-groups and 7-12% Hydroxpropoxy groups.

It is commercially available as METHOCEL™ line of products from DuPont. It is available in a variety of E grades ranging from low to high viscosity.

Especially useful is HPMC of low viscosity grade E3, E5 or E6, corresponding to Viscosity (2%) of 3, 5 or 6 mPa s.

In one embodiment the hydrophilic polymer is incorporated into the fluralaner pyrantel granulation and/or the moxidectin granulation. To bind moxidectin to inert material and control loss of moxidectin during drying, an additional binder (HPMC 5 cps/HPMC E5) was evaluated. Trials were executed with different concentration of HPMC E5 (1 mg, 2 mg & 4 mg).

In one preferred embodiment of the invention and/or embodiments thereof the hydrophilic polymer in the chewable veterinary dosage form may be in the range of from about 1 to about 20 weight %. In an alternative embodiment the amount of the hydrophilic polymer may be in the range of from about 2 to about 15 weight %. One preferred range is from about 5 to about 10 weight %. One preferred range for HPMC is from about 0.1 to 4 weight %.

The stabilizing component preferably comprises of an antioxidant. Antioxidants can also be used to help stabilize the active ingredients in part (compositional granulations and common blend) or whole tablet.

Antioxidants are substances that are used to inhibit oxidation. Antioxidants suitable to be comprised in the present chewable veterinary dosage form include, but are not limited to, ascorbic acid, citric acid, glutathione, tocopherol and its esters, tert-butylhydroquinone (TBHQ), butyl hydroxy anisole (BHA also referred to as 2-tert-butyl-4-hydroxy anisole, 3-tert-butyl-4-hydroxy anisole or a mixture thereof) and butyl hydroxy toluene (BHT also referred as 2,6-di tert-butyl 4-methyl phenol).

In one embodiment the antioxidant was incorporated into the moxidectin granulation. It is preferred that the antioxidant, especially BHT is present in the moxidectin granulation. Butylated hydroxyl toluene protects the moxidectin from oxidative degradation.

In one preferred embodiment of the invention and/or embodiments thereof, the antioxidants comprised in the palatable chewable veterinary dosage form may be in the range of from 0.001 to 1.00 weight %.

The amount of antioxidant in the composition is about 0.01 w/w % to about 0.5 w/w % of the total weight of the tablet. The preferred amount of antioxidant is about 0.05 w/w % to about 0.2 w/w % of the total weight of the tablet.

The more preferred amount antioxidant is about 0.1 w/w % of the total weight of the tablet.

The palatable, chewable dosage form according to the invention additionally comprises at least one flavor. In line with the present application a flavor can be regarded as the sensory impression of the palatable, chewable dosage form. In particular, flavors can affect the senses of taste and smell. In the present case palatable, chewable dosage form is added with a flavor to attract its administration for the animal to be treated. The flavor can be chosen depending on the animal to be treated.

In one preferred embodiment of the invention and/or embodiments thereof the flavor (e) is selected from chicken flavor, pork flavor, beef flavor, ham flavor, fish flavor, vegetarian flavor, Chardex Hickory flavor, artificial flavor, sweet apple & molasses flavor and mixtures thereof, in particular, pork liver flavor.

In one preferred embodiment of the invention and/or embodiments thereof the amount of the flavor comprised in the palatable, chewable dosage form may be in the range of from 2 to 35 weight %. In an alternative embodiment the amount of such a compound may be in the range of from 5 to 30 weight %. The preferred range is from 10 to 25 weight %, especially 5 to 10%. Alternatively, the flavor amount is lower than 2% weight.

It has been surprisingly discovered that with only one flavor present in the palatable, chewable dosage form according to the invention has been shown superior palatability when administered to dogs. In a preferred embodiment the flavor is pork liver flavor.

The palatable, chewable dosage form additionally comprises a carrier comprising one or more veterinary acceptable excipients. The veterinary acceptable excipient includes excipients that are construed as binders, fillers, disintegrants, surfactants, lubricants, glidants, and colorants.

Binders are used to add cohesiveness to the separate granulations and to the final blended composition, thereby providing the necessary bonding to form a cohesive mass and to ensure a suitable compacted tablet form. These binding agents are conventionally used in direct compression tablets and are described in Lieberman et. al., Pharmaceutical Dosage Forms, 2 Ed., Vol. 1, (1990).

Non-limiting examples of veterinary acceptable binders include, but are not limited to: microcrystalline cellulose, carboxymethyl cellulose, sodium carboxy methyl cellulose, hydroxypropyl cellulose (HPC), polyvinylpyrrolidone (e.g., PVP, povidone (Kollidon 25, 30, and 90) and co-povidone (Kollidon VA 64), polyethylene glycol, acacia, corn syrup solids, tragacanth gum, gelatin, carnauba wax, alginate, and mixtures thereof.

The preferred binding agents for the palatable, chewable dosage form are carboxymethyl cellulose, HPC, PVP, polyethylene glycol, corn syrup solids, gelatin, and mixtures thereof.

HPMC can also be considered a binding agent as it does provide some binding qualities to the moxidectin granulation, however, for purposes of this palatable, chewable dosage form, it is included in the components that are included as a stabilizing agent (stabilizer).

The amount of binding agent(s), not including HPMC, in the composition is about 6 to 10 w/w % of the total weight of the tablet. The preferred amount of binding agent(s) in the composition is about 7 to 9 w/w % of the total weight of the tablet.

The palatable, chewable dosage form comprises at least one veterinary acceptable excipient that is a filler. Non-limiting examples of fillers include: starch (e.g., corn, potato, tapioca, and the like), sugars (e.g., lactose, fructose, mannitol), and the like, including hydrous and anhydrous forms), cellulose (e.g., methyl cellulose, microcrystalline cellulose, ethyl cellulose, and the like).

The amount of filler is about 20 to 50 w/w % of the total weight of the composition. The preferred amount of filler is about 40 to 48 w/w % of the total weight of the composition. The more preferred amount of filler is about 42 to 46 w/w % of the total weight of the composition or alternatively 20 to 30%.

The palatable, chewable dosage form comprises at least one veterinary acceptable excipient that is a disintegrant. Disintegrants are compounds which enhance the ability of the tablet, when in contact with a liquid, preferably water, to break into smaller fragments.

Non-exclusive examples of veterinary acceptable disintegrants include: starch including pregelatinized and modified starches, microcrystalline cellulose, methyl cellulose, carboxymethyl cellulose, carboxymethyl cellulose sodium, crospovidone, magnesium aluminum silicate, guar gum, alginic acid, sodium alginate, calcium alginate, chitosan, croscarmellose sodium (e.g., Ac-Di-Sol®), sodium starch glycolate, and the like, and mixtures thereof.

The amount of disintegrant in the composition is about 5 to about 15 w/w % of the total weight of the composition. The preferred amount of disintegrant in the composition is about 8 to 12 w/w % of the total weight of the composition.

Surfactants can be regarded as substances lowering the interfacial tension between two phases. Surfactants may also be added to help solubilize the active drug, to prevent crystallization, and to prevent phase separation.

Common surfactants are alkylsulfates (for example sodium lauryl sulfate), alkyltrimethylammonium salts, alcohol ethoxylates and the like.

In one preferred embodiment of the invention and/or embodiments thereof, the surfactant comprised in the palatable chewable veterinary dosage form is sodium lauryl sulfate and may be in the range of from 0.1 to 10.0 w/w %, preferably about 2 w/w %.

The palatable, chewable dosage form comprises at least one veterinary acceptable excipient that is a glidant and a lubricant.

Glidants, can be used to improve the flowability. Traditionally, talc was used as glidant but is nowadays nearly fully replaced by colloidal silica.

The amount of glidant in the palatable, chewable dosage form is about 0.1 to 0.75 w/w % of the total weight of the tablet. In one preferred embodiment of the invention and/or embodiments thereof, the flow agents comprised in the palatable, chewable dosage form may be in the range of from 0.15 to 0.3 weight %.

Lubricants generally can be regarded as substances which are suitable to reduce friction, such as static friction, sliding friction and rolling friction.

The lubricant is preferably a stearate or fatty acid, more preferably an earth alkali metal stearate, such as magnesium stearate. In one preferred embodiment of the invention and/or embodiments thereof, the lubricants comprised in the palatable, chewable dosage form may be in the range of from 0.1 to 10.0 weight %, preferably 0.2 to 1%.

The palatable, chewable dosage form can comprise at least one veterinary acceptable excipient that is a colorant. Colorants can be added to the composition to enhance its physical appearance.

The amount of colorant in the composition is about 0.1 w/w % to about 2 w/w %, and preferably, about 0.9 w/w % to 1.5 w/w % of the total weight of the tablet.

The palatable, chewable dosage form is prepared using at least one solvent.

The solvent is used for dissolving, suspending, and blending operations to prepare separate granulation components. The granulations are dried prior to subsequent processing, and therefore, the solvents evaporate from the granulation.

However, residual solvents may exist in the final compositional blend and/or compressed tablets. The residual solvents may evaporate further over time. Solvents include water, ethanol, and mixtures thereof.

The palatable, chewable dosage form is prepared by general granulation, blending, milling, sieving, and compression procedures.

A wet granulation process was developed for granulating the moxidectin. Moxidectin is physically separated from the other active ingredients (fluralaner and pyrantel pamoate) during the granulation process thereby providing a physical barrier around the moxidectin particles.

The palatable, chewable dosage form is prepared as follows:
a. an isoxazoline and pyrantel pamoate granulation is prepared by: 1) dry blending the isoxazoline compound and pyrantel pamoate with a disintegrant, filler, colorant and surfactant; 2) preparing a solution of a solvent and a cellulosic polymer; 3) blending the fluralaner and pyrantel pamoate dry blend with the solution to prepare a isoxazoline-pyrantel granulate in a high-shear mixer granulator; 4) dry and mill the isoxazoline-pyrantel granulate.
b. a moxidectin granulation is prepared by: 1) dry blending an porous silica, a filler and an alkalizer, especially a magnesium carbonate; 2) dissolving moxidectin in a solvent with a non-cellulosic polymer and antioxidant; 4) blending the dry blend with the moxidectin solution to prepare a moxidectin granulate in a high-shear mixer granulator; 4) dry and mill the moxidectin granulate.
c. Preparing a final dry blend by: 1) blending flavor with a filler, a disintegrant, a colorant, a glidant to prepare a mixture; 2) blending the isoxazoline-pyrantel pamoate granulation and moxidectin granulation with the mixture; 3) blending the mixture with a lubricant and compressing the blend into finished palatable chewable tablets.

The final compositional blend is a common blend and can be used to prepare tablets as chewable veterinary dosage form of different sizes and shapes with a consistent % w/w distribution of active ingredients. The compressed tablets are packaged.

In one embodiment the palatable, chewable dosage form comprises about 12.5 w/w % of fluralaner. Tablet strengths and sizes contain different amounts of fluralaner. For example, each tablet can contain about 1 mg to about 800 mg of fluralaner. In one aspect, the tablet can contain about 10 mg, 12.5 mg, 15 mg, 20 mg, 25 mg, 30 mg, 40 mg, 50 mg, 60 mg, 70 mg, 75 mg, 80 mg, 90 mg, 100 mg, 150 mg, 200 mg, 300 mg, 400 mg, 500 mg, 600 mg, 700 and about 800 mg of fluralaner.

A preferred amount of fluralaner in the palatable, chewable dosage form is about 25 mg/tablet, 50 mg/tablet, 100 mg/tablet, 200 mg/tablet, 400 mg/tablet, and 600 mg/tablet.

Dosage amounts in the different tablet strengths are used to accommodate animals of different weights such that each animal can receive a dosage of about 1 to 35 mg/kg, preferably about 5 to 25 mg/kg bodyweight fluralaner. In one embodiment the dosage is between 8 and 15 mg/kg bw, especially 10 mg/kg bodyweight.

The palatable, chewable dosage form comprises about 0.03 w/w % (i.e., about 0.031 w/w %) of moxidectin based on the total weight of the tablet. Tablet strengths and sizes contain different amounts of moxidectin. For example, tablets can contain about 0.01 mg to about 3.6 mg of moxidectin. Preferably, tablets can contain about 0.01 mg, 0.03 mg, 0.06 mg, 0.08 mg, 1.0 mg, or about 1.2 mg of moxidectin.

A preferred amount of moxidectin is about 0.0625 mg/tablet, 0.125 mg/tablet, 0.25 mg/tablet, 0.5 mg/tablet, 1 mg/tablet, and 1.5 mg/tablet. Dosage amounts in the different tablet strengths are used to accommodate animals of different weights such that each animal can receive a dosage of about 25 μg/kg, preferably about 0.025 to about 0.05 mg/kg moxidectin per kg bodyweight.

The palatable, chewable dosage form comprises about 18 w/w % of pyrantel pamoate (corresponding to 6.25% pyrantel) based on the total weight of the tablet. The skilled person is able to calculate from the pyrantel amount the necessary pyrantel pamoate amount. Tablet strengths can contain different amounts of pyrantel pamoate. For example, each tablet can contain about 15 mg to about 1000 mg of pyrantel pamoate.

The preferred amount of pyrantel is 12.5 mg/tablet, 25 mg/tablet, 50 mg/tablet, 100. mg/tablet, 200 mg/tablet, and 300 mg/tablet. Dosage amounts in the different tablet strengths are used to accommodate animals of different weights such that each animal can receive a dosage of about 4.0 to about 10.0 mg/kg of pyrantel, preferably about 5 mg/kg bodyweight pyrantel.

The preferred tablet strengths of each active agent (fluralaner, moxidectin, and pyrantel) for the tableted composition includes: 1) about 25 mg fluralaner, 0.0625 mg moxidectin, and 12.5 mg pyrantel base (36 mg pyrantel pamoate); 2) about 50 mg fluralaner, 0.125 mg moxidectin, and 25 mg pyrantel base (72 mg pyrantel pamoate); 3) about 100 mg fluralaner, 0.25 mg moxidectin, and 50 mg pyrantel base (144 mg pyrantel pamoate); 4) about 200 mg fluralaner, 0.50 mg moxidectin, and 100 mg pyrantel (288 mg pyrantel pamoate); 5) about 400 mg fluralaner, 1 mg moxidectin, and 200 mg pyrantel base (576 mg pyrantel pamoate); and 6) about 600 mg fluralaner, 1.5 mg moxidectin, and 300 mg pyrantel base 864 mg pyrantel pamoate).

Tablet weights range from about 200 mg for the 25 mg fluralaner tablet to about 4800 mg for the 600 mg fluralaner tablet.

The palatable, chewable dosage form and different size/strength tablets provide a dosage of about 10 mg/kg to 20 mg/kg fluralaner, about 25 μg/kg to about 50 μg/kg moxidectin, and about 5 mg/kg to about 10 mg/kg pyrantel.

The Hardness of the palatable, chewable dosage form can be measured by practical laboratory or manufacturing tablet hardness tester instruments to determine the breaking point and structural integrity of a tablet. The hardness values of the tablets range from about 20N to about 500N. The tablet hardness values range upward with increasing tablet size. For example, tablet hardness for the 3 mg, 6 mg, 12 mg, 24 mg, 48 mg, and 72 mg tablets ranges from about 30-70N, 40-120N, 60-150N, 100-250N, 140-300N, and 200-400N, respectively.

The respective hard tablet weights are about 200 mg, 400 mg, 800 mg, 1600 mg, 3200 mg, and 4800 mg. The unit of hardness measure, N, is Newtons, the measure of force needed to break the tablet. 1 N is equivalent to 1 kg*m/s².

Hardness is based on a combination of factors, for example, but not limited to tablet shape, surface area, thickness, active agent, excipient(s), and compression forces. Depending on tablet size, the tablet hardness ranges from about 20N to 500N. The unit of force can also be defined in kiloponds (kp) wherein 1 kp=9.80665N.

In an alternative embodiment the granulation with the macrocyclic lactone, especially moxidectin that has been stabilized according to the current invention are used in a process to incorporate moxidectin in the form of a moxidectin granulation in a soft chewable veterinary dosage form.

Therefore, in one alternative embodiment the invention is directed to a palatable soft chewable composition that comprises a moxidectin granulation as described in this application, that has been stabilized as described in this application. Such a palatable soft chewable composition has a hardness that is below the hardness of the compressed tablet described herein. Such soft chewable compositions are known in the art and processes to manufacture such dosage forms by molding or extrusion have been described in the prior art.

The palatable, chewable dosage form of the present invention are especially suitable for combating parasites that infest mammals (including humans). Mammalian subjects include primates (e.g. monkeys), equine (e.g. horses), canine (e.g. dogs), feline (e.g. house cats).

In some embodiments of this invention, the palatable, chewable dosage form is administered to treat parasitoses of an animal (or make a medicament to treat parasitoses of an animal). The term “parasitoses” includes pathologic conditions and diseases associated with or caused by one or more ectoparasites directly, such as for example anemia and flea allergy dermatitis. It also includes pathologic conditions or diseases associated with or caused by one or more vector-transmitted pathogens, such as for example, Lyme disease, Ehrlichiosis (particularly Canine Ehrlichiosis), and Rocky Mountain spotted fever from vector ticks.

This invention also relates to treatment methods wherein at least an ancillary goal of controlling ectoparasites in and/or on an animal is to control an ectoparasitic infestation in an environment that is occupied (periodically or continuously) by the animal. In some such embodiments, for example, the animal is a companion animal (e.g. a cat or a dog). The environment may be, for example, a house or other shelter, a room, a pen, a stall or other confinement means, bedding etc.

### EXAMPLES

The invention is further described by the following compositional example which further illustrates the invention, and is not intended, nor should it be interpreted to, limit the scope of the invention.

Palatable, hard chewable compositions containing fluralaner, moxidectin, and pyrantel pamoate were prepared and evaluated for palatability, stability and pharmacokinetic.

Example 1 describes an example of a stable, palatable composition.

### Example 1. Composition

| Component | Weigh % of chewable tablet |
|---|---|
| Fluralaner | 12.5 |
| Pyrantel pamoate | 18.0 (6.25% Pyrantel) |
| Moxidectin | 0.03 |
| Flavour | 8.0 |
| Filler | 28.75 |
| Disintegrant | 9.0 |
| Colorant | 0.95 |
| Surfactant | 2.0 |
| Non-cellulosic polymer | 18.625 |
| Alkalizer | 1.4 |
| Glidant | 0.2 |
| Lubricant | 0.5 |
| Antioxidant | 0.1 |

A single tablet blend is used for manufacturing of the chewable tablets.

Manufacture of the tablets involves manufacture of granules containing the moxidectin active ingredient and a second granulation containing the isoxazoline, e.g. fluralaner and pyrantel active ingredient.

These two granulations are then blended with the extra-granular materials and compressed.

### Part A: Fluralaner and Pyrantel Granules

Fluralaner, pyrantel pamoate, microcrystalline cellulose, sodium lauryl sulfate, croscarmellose sodium and Pigment blend brown were sieved and mixed in a high shear mixer granulator to form the dry mix.

Hypromellose was dissolved in purified water under stirring to prepare the binder solution. The dry mix was granulated using the binder solution. The wet mass was dried in fluidized bed equipment. Dried granules were milled and sieved.

### Part B—Moxidectin Granules:

Moxidectin solution was prepared by adding Poloxamer 188 slowly to ethanol. Purified water added into it at the ratio of ethanol & water (80:20) (40% w/w) to get clear solution. Dispensed quantity of butylated hydroxy toluene was added into the clear solution. After BHT dissolving, dispensed quantity of moxidectin was added to the solution and stirred until clear solution formed.

Microcrystalline cellulose (Ceolus UF711), Neusilin US2, Light Magnesium carbonate and hypromellose were sieved and mixed in a mixer granulator. Dry mix material was granulated using Moxidectin solution. Wet mix was dried in fluidized bed equipment and moxidectin granules were milled and sieved.

### Part C: Extra-Granular Materials:

Step 1: Part A & part B granules were dispensed as per batch size.

Pigment blend brown, Microcrystalline cellulose, Croscarmellose sodium, pork liver flavor, and Colloidal silicon dioxide were mixed and sieved.

### Blending of Part A & Part B Granules:

Fluralaner and Pyrantel pamoate granules and moxidectin granules were weighed and mixed with extra-granular material and magnesium stearate and final blend prepared for compression. Compressing the blend into finished palatable chewable tablets.

Example Composition 1

| | % w/w |
|---|---|
| Part A Granules | |
| Fluralaner | 12.500 |
| Pyrantel pamoate | 18.000 (6.25% |
| | Pyrantel) |
| Microcrystalline cellulose | 16.400 |
| Croscarmellose sodium | 4.000 |
| Pigment blend brown | 0.600 |
| Sodium lauryl sulfate | 2.000 |
| Hydroxy propyl methyl cellulose | 3.000 |
| Purified water | — |
| Weight of granules | 56.500 |
| Part B Granules | |
| Moxidectin | 0.03125 |
| Butylated hydroxy toluene | 0.100 |
| Poloxamer P188 | 9.375 |
| Magnesium Aluminometasilicate (Neusilin US2) | 6.250 |
| Microcrystalline cellulose (Ceolus UF711) | 6.339 |
| Light magnesium carbonate | 1.405 |
| Ethanol | — |
| Purified water | — |
| Weight of granules | 23.500 |
| Extra-Granular Materials | |
| Microcrystalline cellulose | 5.950 |
| pork liver flavor | 8.000 |
| Croscarmellose sodium | 5.000 |
| Pigment blend brown | 0.350 |
| Colloidal Silicon Dioxide | 0.200 |
| Magnesium Stearate | 0.500 |
| Total | 100.000 |

Example Composition 2

| | % w/w |
|---|---|
| Part A Granules | |
| Fluralaner | 12.500 |
| Pyrantel pamoate | 18.000 (6.25% |
| | Pyrantel) |
| Microcrystalline cellulose | 16.400 |
| Croscarmellose sodium | 4.000 |
| Pigment blend brown | 0.600 |
| Sodium lauryl sulfate | 2.000 |
| Hydroxy propyl methyl cellulose | 3.000 |
| Purified water | — |
| Weight of granules | 56.500 |
| Part B Granules | |
| Moxidectin | 0.031 |
| Butylated hydroxy toluene | 0.100 |
| Poloxamer P188 | 9.375 |
| Magnesium Aluminometasilicate (Neusilin US2) | 6.250 |
| Microcrystalline cellulose (Ceolus UF711) | 6.250 |
| Magnesium carbonate | 1.000 |
| Hydroxypropyl cellulose (HPC) | 1.194 |
| Ethanol | — |
| Purified water | — |
| Weight of granules | 24.200 |
| Extra-Granular Materials | |
| Microcrystalline cellulose | 5.450 |
| pork liver flavor | 8.000 |
| Croscarmellose sodium | 5.000 |
| Pigment blend brown | 0.350 |
| Colloidal Silicon Dioxide | 0.200 |
| Magnesium Stearate | 0.300 |
| Total | 100.000 |

Example Composition 3

| | % w/w |
|---|---|
| Part A Granules | |
| Fluralaner | 12.500 |
| Pyrantel pamoate | 18.000 (6.25% Pyrantel) |
| Microcrystalline cellulose | 12.400 |
| Croscarmellose sodium | 4.000 |
| Pigment blend brown | 0.600 |
| Sodium lauryl sulfate | 2.000 |
| pork liver flavor | 4.000 |
| Hydroxy propyl methyl cellulose | 3.000 |
| Weight of granules | 56.500 |
| Part B Granules | |
| Moxidectin | 0.031 |
| Butylated hydroxy toluene | 0.100 |
| Poloxamer P188 | 6.250 |
| Magnesium Aluminometasilicate | 6.250 |
| (Neusilin US2) | |
| Microcrystalline cellulose (Ceolus UF711) | 6.250 |
| Magnesium carbonate (dispersion) | 0.450 |
| Hydroxy propyl methyl cellulose | 0.25 |
| Weight of granules | 19.58 |
| Extra-Granular Materials | |
| Microcrystalline cellulose | 9.87 |
| pork liver flavor | 8.00 |
| Croscarmellose sodium | 5.00 |
| Pigment blend brown | 0.35 |
| Colloidal Silicon Dioxide | 0.20 |
| Magnesium Stearate | 0.50 |
| Total | 100.00 |

Example Composition 4

| | % w/w |
|---|---|
| Part A Granules | |
| Fluralaner | 12.50 |
| Pyrantel pamoate | 18.00 (6.25% |
| | Pyrantel) |
| Microcrystalline cellulose | 16.40 |
| Croscarmellose sodium | 5.00 |
| Pigment blend brown | 0.60 |
| Sodium lauryl sulfate | 2.00 |
| Hydroxy propyl methyl cellulose | 3.00 |
| Weight of granules | 57.50 |
| Part B Granules | |
| Moxidectin | 0.031 |
| Butylated hydroxy toluene | 0.100 |
| Poloxamer P188 | 9.375 |
| Magnesium Aluminometasilicate (Neusilin US2) | 6.250 |
| Microcrystalline cellulose (Ceolus UF711) | 6.250 |
| Pigment Blend Brown | 0.050 |
| Hydroxy propyl cellulose (HPC) | 1.194 |
| Weight of granules | 23.25 |
| Extra-Granular Materials | |
| Microcrystalline cellulose | 5.40 |
| pork liver flavor | 8.00 |
| Croscarmellose sodium | 5.00 |
| Pigment blend brown | 0.35 |
| Colloidal Silicon Dioxide | 0.20 |
| Magnesium Stearate | 0.30 |
| Total | 100.00 |

Example Composition 5

| | % w/w |
|---|---|
| Part A Granules | |
| Fluralaner | 12.50 |
| Pyrantel pamoate | 18.00 (6.25% |
| | Pyrantel) |
| Microcrystalline cellulose | 16.40 |
| Croscarmellose sodium | 4.00 |
| Pigment blend brown | 0.60 |
| Sodium lauryl sulfate | 2.00 |
| Hydroxy propyl methyl cellulose | 3.00 |
| Weight of granules | 56.50 |
| Part B Granules | |
| Moxidectin | 0.03 |
| HP-β-Cyclodextrin | 1.25 |
| Butylated hydroxy toluene | 0.10 |
| Magnesium Aluminometasilicate (Neusilin US2) | 6.42 |
| Microcrystalline cellulose (Ceolus UF711) | 5.00 |
| Croscarmellose sodium | 2.50 |
| Pigment Blend Brown | 0.20 |
| Hydroxy propyl methyl cellulose | 2.00 |
| Ethanol + Water (90:10) | — |
| Weight of granules | 17.50 |
| Extra-Granular Materials | |
| Microcrystalline cellulose | 7.30 |
| pork liver flavor | 8.00 |
| Croscarmellose sodium | 5.50 |
| Pigment blend brown | 0.20 |
| Sodium Chloride | 1.00 |
| Brewer's Yeast | 3.50 |
| Magnesium Stearate | 0.50 |
| Total | 100.00 |

Example Composition 6

| | % w/w |
|---|---|
| Part A Granules | |
| Fluralaner | 12.50 |
| Pyrantel pamoate | 18.00 (6.25% Pyrantel) |
| Microcrystalline cellulose | 16.40 |
| Croscarmellose sodium | 4.00 |
| Pigment blend brown | 0.60 |
| Sodium lauryl sulfate | 2.00 |
| Hydroxy propyl methyl cellulose | 3.00 |
| Purified water | — |
| Weight of granules | 56.50 |
| Part B Granules | |
| Moxidectin | 0.03 |
| Butylated hydroxy toluene | 0.10 |
| Poloxamer P188 | 9.74 |
| Magnesium carbonate | 0.13 |
| Ethanol | — |
| Purified water | — |
| Weight of granules | 10.00 |
| Extra-Granular Materials | |
| Microcrystalline cellulose | 14.80 |
| pork liver flavor | 8.00 |
| Croscarmellose sodium | 5.50 |
| Sodium Chloride | 1.00 |
| Brewer's Yeast | 3.50 |
| Pigment blend brown | 0.20 |
| Magnesium Stearate | 0.50 |
| Total | 100.00 |

### Example 2: Evaluation of Palatability

Objective: To determine the oral acceptability of different flavored chewable tablets in Beagle-type dogs.

### Test Items:

Three different placebo chewable tablets (A, F, G).
Tablet A included pork liver flavor at 8%;
Tablet F included 12% pork liver flavor with no Brewer's yeast or NaCl;
Tablet G included 8% pork liver flavor with no Brewer's yeast or NaCl.

Tablets F and G included the alkalizer magnesium carbonate at 1% (equivalent to 4% magnesium carbonate in the moxidectin granules).

The placebo chewable tablets were prepared analogous to the process described in this application.

TABLE 1
Composition test formulation

| | Tablet A | | |
|---|---|---|---|
| | (comparative) | Tablet F | Tablet G |
| Test Article | % w/w | % w/w | % w/w |
| Flavor | 8.0 | 12.0000 | 8.0000 |
| | Pork liver | Pork Liver | Pork Liver |
| | flavor | flavor | flavor |
| Lactose Monohydrate | 68.9 | — | — |
| Microcrystalline Cellulose | — | 38.0000 | 38.25 |
| HPMC E5 | 3.3 | 3.0000 | 3.0000 |
| Croscarmellose Sodium | 10.0 | 9.0000 | 9.0000 |
| Butylated Hydroxy Toluene | 0.2 | 0.1000 | 0.1000 |
| Poloxamer 188 | 2.8 | 9.3750 | 9.3750 |
| Sodium Lauryl Sulfate | 0.5 | 2.0000 | 2.0000 |
| NaCl | 1.0 | — | — |
| Brewer's Yeast | 3.5 | — | — |
| Magnesium Carbonate | — | 1.0000 | 1.0000 |
| Magnesium | — | 6.2500 | 6.2500 |
| Aluminometasilicate | | | |
| (Neusilin) | | | |
| Microcrystalline | — | 6.2825 | 6.2825 |
| Cellulose (Ceolus) | | | |
| Pigment blend brown | 1.3 | 0.9500 | 0.9500 |
| Hydroxypropyl Cellulose | — | 1.1925 | 1.1925 |
| (HPC) | | | |
| Microcrystalline Cellulose | — | 10.3500 | 14.1000 |
| Colloidal Silicon Dioxide | 0.2 | 0.20000 | 0.20000 |
| Magnesium Stearate | 0.3 | 0.3000 | 0.3000 |
| Total | 100.0 | 100.0000 | 100.0000 |

30 dogs were included in the study (10 dogs per group). The study ran for 6 days, and the dogs were offered 3 different flavored chewable tablets (Formulations A, F, G) on 2 consecutive days each. The acceptability was determined based on the chewable tablet being completely consumed, partially consumed, or not consumed at all.

### Results:

The percentage acceptability was 100% for all chewable tablet formulations. This study demonstrated that the alkalizer magnesium carbonate has no effect on palatability and the lower level of flavor was equally effective.

Conclusion: Palatability above 80% was demonstrated for all of the tested flavored chewable tablets. Addition of the alkalizer magnesium carbonate has no impact on palatability and that a single palatability agent is sufficient to reach very high palatability in dogs.

### Example 3

### Pharmacokinetic Profile Following Oral Administration of Chewable Formulations of Isoxazoline, Moxidectin, and Pyrantel Combinations

The objective of this study was to compare the blood plasma pharmacokinetic profile of a compressed tablet containing fluralaner, moxidectin, and pyrantel according to the invention as described in Table 3 to a commercially available combination chewable tablet Simparica Trio (Zoetis), and an fluralaner only chewable formulation after a single oral administration in dogs.

STUDY DESIGN: The test compounds were administered orally to five beagle dogs per dose group for a total of fifteen dogs.

TABLE 2
Composition of the test formulation according to the invention

| S. No. | Ingredients | % w/w |
|---|---|---|
| Intra-granular materials | | |
| Part-A | | |
| 1 | Fluralaner | 12.500 |
| 2 | Pyrantel pamoate | 18.000 |
| 3 | Microcrystalline cellulose | 16.400 |
| 4 | Croscarmellose sodium | 4.000 |
| 5 | Pigment blend brown | 0.600 |
| 6 | Sodium lauryl sulfate | 2.000 |
| 7 | Hydroxy propyl methyl cellulose | 3.000 |
| 8 | Purified water | — |
| | Total part A | 56.500 |
| Part-B | | |
| 9 | Moxidectin | 0.031 |
| 10 | Butylated hydroxy toluene | 0.100 |
| 11 | Poloxamer P188 | 9.375 |
| 12 | Magnesium Aluminometasilicate (Neusilin US2) | 6.250 |
| 13 | Microcrystalline cellulose (Ceolus UF711) | 6.339 |
| 14 | Light magnesium carbonate | 1.405 |
| 15 | Ethanol | — |
| 16 | Purified water | — |
| | Total part B | 23.500 |
| Extra-granular material | | |
| 17 | Microcrystalline cellulose | 5.950 |
| 18 | pork liver flavor | 8.000 |
| 19 | Croscarmellose sodium | 5.000 |
| 20 | Pigment blend brown | 0.350 |
| 21 | Aerosil 200 Pharma | 0.200 |
| 22 | Magnesium Stearate | 0.500 |
| | | 20.000 |
| | Total weight | 100.000 |

TABLE 3
Study design

| | No. | Isoxazoline | Moxidectin | Pyrantel | |
|---|---|---|---|---|---|
| Treatment | of | Dose | Dose | dose | Formulation |
| ID | dogs | (mg/kg) | (mg/kg) | (mg/kg) | Type^{2, 3} |
| 1 | 5 | 10 | 0.025 | 5 | Test |
| | | (fluralaner) | | | formulation |
| 2 | 5 | 1.2 | 0.024 | 5 | Competitor |
| | | (sarolaner) | | | tablet |
| 3 | 5 | 10 | 0 | 0 | Control Chew |
| | | (fluralaner) | | | |

Each animal was administered the chewable tablet or chew by placing it in the back of the oral cavity over the tongue to initiate swallowing.

Plasma was obtained from the collected blood samples and analyzed for concentrations of the test compounds (R-fluralaner, S-fluralaner, moxidectin, and pyrantel). Individual blood samples (approximately 3 mL per sample) were taken via jugular or cephalic venipuncture into EDTA tubes from all dogs for analysis. Blood samples were collected at the following time points: pretreatment (within 2 h prior to dosing) and approximately 2, 4, 8 (±15 min), 24, 48 (±30 min), 72, 168, 336, and 720 (±60 min) hours after dosing.

### Results:

### Fluralaner

The AUCₜₗₐₛₜ, Tₘₐₓ, and half-life were comparable for all treatment groups.

### Moxidectin

The Cₘₐₓ was slightly higher for the competitor tablet relative to the test formulation.
The AUCₜₗₐₛₜ was slightly higher for the test formulation relative to the competitor.
The Tₘₐₓ was comparable for all treatment groups.
The half-life was higher for the test formulation relative to the comparator.
Pyrantel The Cₘₐₓ was slightly higher for the test formulation relative to the competitor tablet.
The AUCₜₗₐₛₜ, was slightly higher for the test formulation relative to the competitor tablet.
The Tₘₐₓ, and half-life were comparable for all treatment groups.

TABLE 4
Fluralaner comparison

| Pharmacokinetic | Control | | | |
|---|---|---|---|---|
| Parameter | fluralaner Chew | | Test formulation | |
| Dose(mg/kg) | 25 | | 25 | |
| Tₘₐₓ (hour) | | | | |
| Total fluralaner | 4 | | 4 | |
| Cₘₐₓ (ng/mL) | | | | |
| R-fluralaner | 1822 | (±233) | 2650 | (±378) |
| S- Fluralaner | 2976 | (±411) | 3598 | (±492) |
| Total fluralaner | 4664 | (±599) | 6240 | (±595) |
| AUC_{(0-168 h)} (h*ng/mL) | | | | |
| R-fluralaner | 207180 | (±46172) | 229598 | (±14621) |
| S- Fluralaner | 326890 | (±70677) | 331102 | (±53024) |
| Total fluralaner | 534070 | (±113713) | 560700 | (±61228) |

TABLE 5
Moxidectin and Pyrantel comparison

| Pharmacokinetic | Control Commercial tablet | | | | Test formulation | | | |
|---|---|---|---|---|---|---|---|---|
| Parameter | Moxidectin | | Pyrantel | | Moxidectin | | Pyrantel | |
| Dose (mg/kg) | 0.024 | | 5 | | 0.025 | | 5 | |
| Tₘₐₓ (hour) | 2 | | 2 | | 4 | | 2 | |
| Cₘₐₓ (ng/mL) | 37 | (±6) | 162 | (±53) | 27 | (±4) | 212 | (±33) |
| AUC_{(0-168 h)} | 554 | (±102) | 1008 | (±262) | 632 | (±67) | 1227 | (±219) |
| (h*ng/mL) | | | | | | | | |

Given the known relative instability of moxidectin in the presence of agents that might promote acid hydrolysis and give rise to 23-keto nemadectin and 23-Z moxidectin, its two most commonly observed degradation products, an effort was made to optimize the formulation in such a way as to minimize this effect.

As moxidectin is sensitive to degradation, different combinations of polymers, pH modifiers, complexing agent, and adsorbents were evaluated to further protect moxidectin and mitigate stability challenges. The polymers included poloxamer 188 (P188) and hydroxypropylmethyl cellulose (HPMC); the complexing agent included HP-β-cyclodextrin; the adsorbents included magnesium aluminometasilicate and porous microcrystalline cellulose; and the pH modifying agents included sodium citrate, magnesium carbonate, meglumine, arginine, and tromethamine.

For the moxidectin granulation, different combinations of polymers, adsorbents and alkalizers were screened. The polymers include HP-β-Cyclodextrin, Poloxamer 188 (P188), and hydroxypropylmethyl cellulose (HPMC); the absorbents include magnesium aluminometasilicate (Neusilin US2) and microcrystalline cellulose (Ceolus UF711); the alkalizers include sodium citrate, magnesium carbonate, meglumine, arginine, and tromethamine.

The moxidectin granules were blended with the fluralaner-pyrantel granules and the addition of at least one veterinary acceptable excipient, compressed, and placed on accelerated (40° C./75% RH) stability.

From these studies, moxidectin was found to significantly degrade without the use of a stabilizing component that comprises veterinary acceptable excipients selected from the group consisting of an inorganic alkalizer, porous silica and mixtures thereof. Therefore an aspect of the current invention is the use of the stabilizing component that comprises veterinary acceptable excipients selected from the group consisting of an hydrophilic polymer, inorganic alkalizer, porous silica and mixtures thereof in the moxidectin granulation process provided stability to the moxidectin.

The combination of Poloxamer 188 (P188), magnesium aluminometasilicate (Neusilin US2) and magnesium carbonate was found to be the most effective in stabilizing moxidectin.

The combination of Poloxamer 188 (9.375%)+Neusilin US2 (6.250%)+magnesium carbonate (0.5-1% in tablet) was found to be the most effective in stabilizing moxidectin compared with HP-β-Cyclodextrin (1.00%)+Neusilin US2 (8.47%) and HPMC (2.00%)+sodium citrate (0.75%) at 2 months.

A high Poloxamer: Moxidectin (288:1) solution with magnesium carbonate (4% in moxidectin granules) added as a dried form in the premix and a low Poloxamer: Moxidectin (200:1) solution adsorbed on Neusilin-Ceolus and alkalized with magnesium carbonate (2% in moxidectin granules) were then assessed to determine the effect on moxidectin stability in compressed tablets. At 3 months accelerated stability, these combinations were found to be effective in stabilizing moxidectin with low levels of degradants (0.6 for keto-moxidectin and 0.7 for Z-moxidectin) in the tablet.

Although low levels of degradants were achieved, an optimized level of magnesium carbonate was further assessed. The best stability was achieved with a high Poloxamer: Moxidectin (288:1) solution (18.75% Poloxamer in moxidectin granules) and an optimized level of magnesium carbonate added as a dried form in the premix. The amount of magnesium carbonate in the moxidectin granulation is about 2 to 6% w/w; the preferred amount is about 4-6% w/w; and the most preferred amount is about 6% w/w.

A 6-month stability study of compressed tablets manufactured with Poloxamer, Neusilin, and magnesium carbonate showed a total moxidectin degradation of about 2-4% at accelerated stability.

In the final composition, the amount of magnesium carbonate is about 0.5 to 1.5% w/w of the total tablet weight; and more preferably about 1.0 to 1.5% w/w of the total tablet weight; and even more preferably, about 1.2-1.5% w/w of the total tablet weight.

To further minimize moxidectin degradation, the antioxidant, BHT was added to the moxidectin granulation. The amount of BHT in the moxidectin granulation is about 0.1 to 1.0% w/w; preferably about 0.2 to 0.8% w/w; more preferably about 0.3 to 0.6% w/w; and most preferred about 0.3 to 0.5% w/w, which accounts for about 0.1% w/w of the total tablet weight.

In one stability study, moxidectin stability at 25° C./60% RH and 40° C./75% RH in the final compressed tablets is shown below in Table 1.

In another stability study, moxidectin stability at 30° C./65% RH and 40° C./75% RH in the final compressed tablets is shown in Table 2. Stability results represent the initial/6-month results.

The 25 mg-F tablet represents 25 mg fluralaner, 0.0625 mg moxidectin, and 12.5 mg pyrantel. The 50 mg-F tablet represents 50 mg fluralaner, 0.125 mg moxidectin, and 25 mg pyrantel. The 100 mg-F tablet represents 100 mg fluralaner, 0.25 mg moxidectin, and 50 mg pyrantel. The 200 mg-F tablet represents 200 mg fluralaner, 0.50 mg moxidectin, and 100 mg pyrantel. The 400 mg-F tablet represents 400 mg fluralaner, 1 mg moxidectin, and 200 mg pyrantel. The 600 mg-F tablet represents 600 mg fluralaner, 1.5 mg moxidectin, and 300 mg pyrantel.

TABLE 6
Moxidectin Assay Results of Compressed Tablets
at Initial/6 months from Technical Batch (% LC)

| Tablet | 25 mg-F | 50 mg-F | 100 mg-F | 200 mg-F | 400 mg-F | 600 mg-F |
|---|---|---|---|---|---|---|
| 25° C./60% RH | 96.4/95.1 | 96.3/94.3 | 97.3/95.6 | 97.9/97.8 | 96.5/95.0 | 98.0/96.7 |
| 40° C./75% RH | 96.4/91.6 | 96.3/92.5 | 97.3/93.0 | 97.9/95.8 | 96.5/93.2 | 98.0/94.1 |

TABLE 7
Moxidectin Assay Results of Compressed Tablets
at Initial/6 months from Technical Batch (% LC)

| Tablet | 25 mg-F | 600 mg-F |
|---|---|---|
| 30° C./65% RH | 97.5/96.2 | 97.1/95.8 |
| 40° C./75% RH | 97.5/93.4 | 97.1/94.7 |

Stability Results for Poloxamer 188 Level Trials at 40° C./75% RH

Trials were conducted to evaluate the impact of different concentrations of poloxamer 188 on the stability of moxidectin. Concentrations of 0% w/w (no poloxamer), 4.688% w/w, and 11.25% w/w were assessed in the formulation. Trial details are provided in the Table 8.

| | | 9.375% Poloxamer 188 | | | | | | 4.668% Poloxamer 188 | | | 11.250% Poloxamer 188 | | |
|---|---|---|---|---|---|---|---|---|---|---|---|---|---|
| | | w/w of Tablet Weight | | | 0% Poloxamer 188 | | | w/w of Tablet Weight | | | w/w of Tablet Weight | | |
| Test | | Initial | 1 mo | 3 mo | Initial | 1 mo | 3 mo | Initial | 1 mo | 3 mo | Initial | 1 mo | 3 mo |
| Moxidectin Assay, | | 97.4 | 94.6 | 94.6 | 86.1 | 71.4 | 64.5 | 106.5 | 107.8 | 106.7 | 103.2 | 100.8 | 98.3 |
| % LC | | | | | | | | | | | | | |
| (%) | 23-keto | ND | 0.3 | 0.6 | 2.5 | 14.2 | 19.5 | ND | 0.5 | 0.8 | ND | 0.6 | 0.9 |
| | nemadectin | | | | | | | | | | | | |
| | 23-Z | 0.3 | 0.3 | 0.7 | 0.8 | 2.7 | 3.8 | 0.3 | 0.4 | 0.7 | 0.3 | 0.4 | 0.7 |
| | moxidectin | | | | | | | | | | | | |
| | Impurity D^{a} | ND | ND | ND | ND | ND | ND | ND | ND | ND | ND | ND | ND |
| | Impurity E^{b} | ND | ND | ND | ND | ND | ND | ND | ND | ND | ND | ND | ND |

| | | | | | | | | | | | | |
|---|---|---|---|---|---|---|---|---|---|---|---|---|
| Fluralaner Assay, | 101.6 | 101.1 | 102.6 | 102.7 | NT | NT | 99.9 | NT | NT | 102.0 | NT | NT |
| % LC | | | | | | | | | | | | |
| Pyrantel Pamoate | 101.8 | 102.0 | 103.4 | 102.1 | NT | NT | 99.3 | NT | NT | 101.7 | NT | NT |
| Assay, % LC | | | | | | | | | | | | |

The formulation without poloxamer showed a significant drop in moxidectin assay (86.1% LC) and high related substance levels (2.5% 23 keto nemadectin; 0.8% 23Z moxidectin) at the initial time point, followed by a rapid loss of moxidectin during storage. The results provide evidence that poloxamer 188 is beneficial in controlling moxidectin degradation.

1. A palatable chewable veterinary dosage form in the form of a compressed tablet comprising an isoxazoline compound of Formula (I) 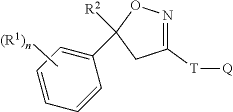
wherein
R¹ is halogen, CF₃, OCF₃, CN,
n is an integer from 0 up to and including 3, preferably 1, 2 or 3,
R² is C₁-C₃-haloalkyl, preferably CF₃ or CF₂Cl,
T is a 5 to 12 membered mono or bicyclic ring system, which is optionally substituted by one or more radicals Y,
Y is methyl, halomethyl, halogen, CN, NO₂, NH₂—C═S, or two adjacent radicals Y form together a chain, especially a three or four-membered chain;
Q is X—NR³R⁴, NR⁵—NR⁶—X—R³, X—R³ or a 5-membered N-heteroaryl ring, which is optionally substituted by one or more radicals;
X is CH₂, CH(CH₃), CH(CN), CO, CS,
R³ is hydrogen, methyl, haloethyl, halopropyl, halobutyl, methoxymethyl, methoxyethyl, halomethoxymethyl, ethoxymethyl, haloethoxymethyl, propoxymethyl, ethylaminocarbonylmethyl, ethylaminocarbonylethyl, dimethoxyethyl, propynylaminocarbonyl-methyl, N-phenyl-N-methyl-amino, haloethylaminocarbonylmethyl, haloethylaminocarbonylethyl, tetrahydrofuryl, methylaminocarbonylmethyl, (N,N-dimethylamino)-carbonylmethyl, propylaminocarbonylmethyl, cyclopropylaminocarbonylmethyl, propenylaminocarbonylmethyl, halo-ethylaminocarbonylcyclopropyl, alkylsulfanyl, alkylsufinalkyl, alkylsulfonalkyl, cycloalkyl 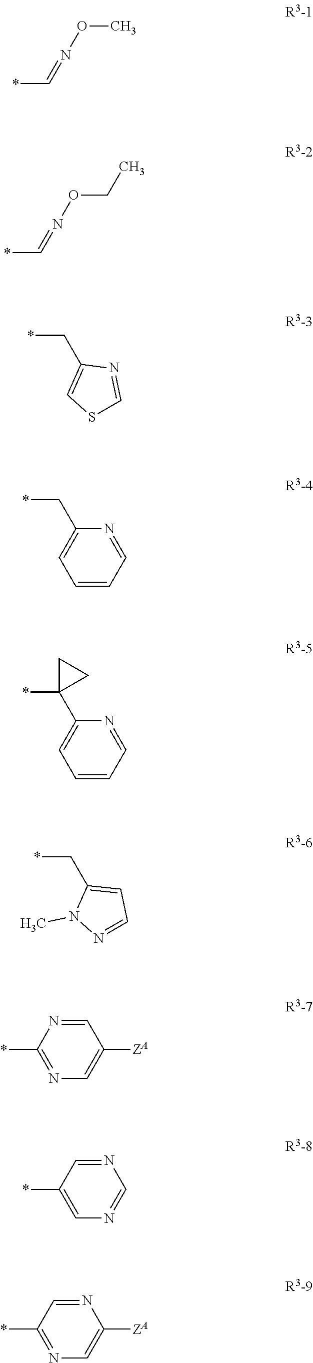 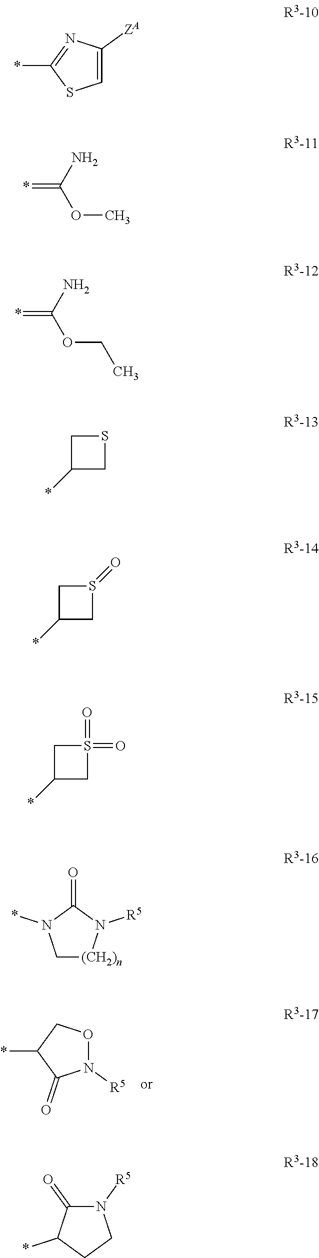
wherein Z^{A} is hydrogen, halogen, cyano, halomethyl, preferably CF₃;
R⁴ is hydrogen, ethyl, methoxymethyl, halomethoxymethyl, ethoxymethyl, haloethoxy-methyl, propoxymethyl, methylcarbonyl, ethylcarbonyl, propylcarbonyl, cyclopropylcarbonyl, methoxycarbonyl, methoxymethylcarbonyl, aminocarbonyl, ethylaminocarbonylmethyl, ethylaminocarbonylethyl, dimethoxyethyl, propynylaminocarbonylmethyl, haloethylaminocarbonylmethyl, cyanomethylaminocarbonylmethyl, or haloethyl-aminocarbonylethyl;
R⁵ is hydrogen, alkyl or haloalkyl;
R⁶ is hydrogen, alkyl or haloalkyl;
or R³ and R⁴ together form a substituent selected from the group consisting of: 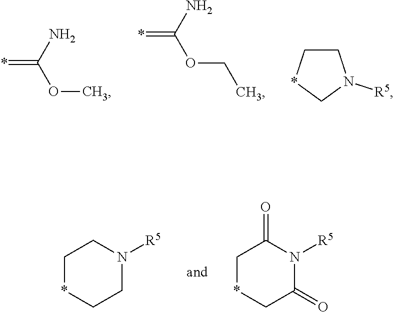
or a salt or solvate thereof, pyrantel pamoate and a macrocyclic lactone compound and a carrier comprising at least one flavor and a stabilizing component that comprises a magnesium carbonate, porous silica or mixtures thereof.

2. The palatable chewable veterinary dosage form of claim 1, characterized in that the macrocyclic lactone is selected from moxidectin and milbemycin oxime, preferably moxidectin.

3. The palatable chewable veterinary dosage form of any one of claims 1 to 2, characterized in that the compressed tablet comprises about 1 to about 4% w/w of the magnesium carbonate.

4. The palatable chewable veterinary dosage form of any one of claims 1 to 3, characterized in that the porous silica is Magnesium Aluminometasilicate.

5. The palatable chewable veterinary dosage form of any one of claims 1 to 4, characterized in that the compressed tablet comprises about 2 to about 10% w/w of the porous silica.

6. The palatable chewable veterinary dosage form of any one of claims 1 to 5, characterized in that the stabilizing component additionally comprises at least one Poloxamer, more preferably Poloxamer P 188.

7. The palatable chewable veterinary dosage form of any one of claims 1 to 6, characterized in that the compressed tablet comprises about 2 to about 15% w/w of the poloxamer.

8. The palatable chewable veterinary dosage form of any one of claims 1 to 7, characterized in that the that the stabilizing component additionally comprises an antioxidant, preferably Butylated hydroxyl toluene (BHT).

9. The palatable chewable veterinary dosage form of any one of claims 1 to 8, characterized in that the compressed tablet comprises about 0.05 to about 2% w/w of the antioxidant.

10. The palatable chewable veterinary dosage form of any one of claims 1 to 9, characterized in that the stabilizing component comprises a combination of at least one magnesium carbonate, adsorbent component, hydrophilic polymer, such as poloxamer and antioxidant.

11. The palatable chewable veterinary dosage form of any one of claims 1 to 10 characterized in that the isoxazoline compound of Formula (I) is fluralaner, afoxolaner, esafoxolaner, sarolaner or lotilaner.

12. The palatable chewable veterinary dosage form of claim 11, characterized in that the isoxazoline compound of Formula (I) is fluralaner.

13. The palatable chewable veterinary dosage form of any one of claims 1 to 12, characterized in that the composition comprises one flavor, preferably a natural flavor, more preferably pork liver flavor.

14. A process for preparing the palatable chewable veterinary dosage form according to any one of claims 1 to 13, characterized in that:
a. A isoxazoline and pyrantel pamoate granulation is prepared by: 1) dry blending the isoxazoline compound of Formula (I) and pyrantel pamoate with disintegrant, filler, colorant and surfactant; 2) preparing a solution of a solvent and a cellulosic polymer; 3) blending the fluralaner and pyrantel pamoate dry blend with the solution to prepare a isoxazoline-pyrantel granulate in a high-shear mixer granulator; 4) dry and mill the isoxazoline-pyrantel granulate.
b. a moxidectin granulation is prepared by: 1) dry blending, porous silica, filler and a magnesium carbonate; 2) dissolving moxidectin in a solvent with non-cellulosic polymer and antioxidant; 4) blending the dry blend with the moxidectin solution to prepare a moxidectin granulate in a high-shear mixer granulator; 4) dry and mill the moxidectin granulate.
c. Preparing a final dry blend by: 1) blending flavor with filler, disintegrant, colorant, glidant to prepare a mixture; 2) blending the isoxazoline-pyrantel pamoate granulation, and moxidectin granulation with the mixture; 3) blending the mixture with a lubricant and compressing the blend into finished palatable chewable tablets.

15. The palatable chewable veterinary dosage form according to any one of claims 1 to 13 for use in the treatment or prevention of parasite infestation of non-human animals.
